(19) United States
(12) Patent Application Publication
(10) Pub. No.: US 2022/0112538 A1
(43) Pub. Date: Apr. 14, 2022

(54) SPECTROSCOPIC METHODS, REAGENTS AND SYSTEMS TO DETECT, IDENTIFY, AND CHARACTERIZE BACTERIA FOR ANTIMICROBIAL SUSCEPTIBLITY

(71) Applicant: Spectral Platforms, Inc., Monrovia, CA (US)

(72) Inventor: Ravi Verma, La Canada, CA (US)

(21) Appl. No.: 17/354,874

(22) Filed: Jun. 22, 2021

Related U.S. Application Data
(60) Provisional application No. 63/042,875, filed on Jun. 23, 2020.

(51) Int. Cl. C12Q 1/04; C12Q 1/18; G01N 21/31

(52) U.S. Cl. CPC C12Q 1/04; G01N 21/31; C12Q 1/18

(57) ABSTRACT

Provided are methods of separating an absorption spectrum into a Rayleigh scattering contribution and an absorption contribution. By performing such separations and removing the influence of Rayleigh scattering, the absorption of a sample can be more accurately measured. Provided are additional methods that involve separating an absorption spectrum into a Rayleigh scattering contribution and an absorption contribution: assessing whether or not a microorganism is present in a biological fluid, assessing the effect of a pharmaceutical drug on a microorganism, and treating a subject suspected of having an infection. Provided are systems and non-transitory computer readable storage media for separating an absorption spectrum into a Rayleigh scattering and an absorption contribution.

### CROSS-REFERENCE TO RELATED APPLICATIONS

This application claims the benefit of U.S. Provisional Patent Application No. 63/042,875 filed on Jun. 23, 2020, the disclosure of which is herein incorporated by reference in its entirety.

### INTRODUCTION

Absorbance spectroscopy with UV or visible light has been used for many biotechnology applications, including the detection of microorganisms such as bacteria. In such procedures, a sample suspected of having bacteria can be contacted with an indicator compound that changes its UV or visible absorption spectrum depending on the presence of a bacterial metabolic product. For example, since some bacteria give off acidic or alkaline metabolic products, the sample can be contacted with a pH indicator. If certain bacteria are present, the pH of the surrounding medium will change, resulting in a color change of the pH indicator, which can then be detected. In contrast, if no bacteria are present, the pH will remain constant and no change in absorption spectra will occur.

Such methods have also been used for other, related purposes. For instance, the type or identity of a bacteria can determine by employing particular indicators. For example, the bacteria H. pylori is capable of producing a urease enzyme that hydrolyzes urea to ammonia, thereby raising the pH of the surrounding fluid. This pH change can then be detected by observing a change in the absorption spectrum of the pH indicator compound.

In other instances, similar methods can be used to characterize the antimicrobial susceptibility response of a bacteria to a particular antibiotic. If exposure to the antibiotic kills all the bacteria, no additional bacterial metabolic products will be generated, and pH and absorption spectrum will not change. In contrast, if the antibiotic fails to kill the bacteria, the pH and absorption spectrum will continue to change as the bacteria metabolize.

However, the experimentally measured absorption spectrum of a test sample is not a perfect representation of the absorption spectrum of the indicator. Instead, the experimentally measured absorption spectrum also includes contributions from elements including Rayleigh scattering and absorption by other components in the test sample. The magnitude of Rayleigh scattering varies depending on the wavelength of light being scattered.

### SUMMARY

Provided are methods of separating an absorption spectrum into a Rayleigh scattering contribution and an absorption contribution. By performing such separations and removing the influence of Rayleigh scattering, the absorption of a sample can be more accurately measured. Provided are additional methods that involve separating an absorption spectrum into a Rayleigh scattering contribution and an absorption contribution: assessing whether or not a microorganism is present in a biological fluid, assessing the effect of a pharmaceutical drug on a microorganism, and treating a subject suspected of having an infection. Provided are systems and non-transitory computer readable storage media for separating an absorption spectrum into a Rayleigh scattering and an absorption contribution.

Aspects of the present disclosure include hydrophobic ligand-albumin complexes, and methods of making and using the same, such as for delivery vehicle for targeting a hydrophobic molecule to a microorganism, and may find use in the detection, e.g., optical detection, of microorganisms in a sample and in the formulation of therapeutic compositions containing hydrophobic active agents, e.g., hydrophobic antibacterial or antifungal agents, for administration to an individual in need thereof. Some of the specific embodiments described herein allow the use of these methods, combined with the rapid detection, identification and antimicrobial susceptibility characterization of bacteria.

Aspects of the present disclosure include products and processes used to determine the presence of bacteria in a sample and includes a culture medium which may be used in products and processes to allow early detection and count of coliform bacteria. The bacterial culture medium which facilitates the early detection and count of coliform bacteria is a mixture of tryptose, lactose, sodium chloride, bile salts, guar gum and an excess amount of phenol red sufficient to provide a high concentration of phenol red in close proximity to the growing bacteria in order to allow detection and count of the growing bacteria in less than 12 hours. Phenol red has a color output that depends on its charge state, and its charge state is altered by the presence of acidic (or alkaline) metabolic byproducts produced by certain bacteria. Most bacteria produce acids under normal metabolic conditions, and acid production results in a decrease in the phenol red peak at 560 nm. Certain embodiments described herein provide for the use of these methods, combined with the detection of bacteria in less than 2-6 hours, and for the characterization of its antimicrobial susceptibility.

Aspects of the present disclosure include bacterial detection methods that characterize an increase in pH (e.g., associated with urea hydrolysis). For instance, described are products and processes used to determine the presence of bacteria (e.g., H. pylori) that is capable of producing a urease enzyme. This enzyme production (due to the presence of H. pylori in a test sample) results in urea (supplied in the media) being hydrolyzed to ammonia, which increases pH (which is distinct from the decrease in pH associated with normal metabolic activity. This results in an increase in the phenol red peak at 560 nm, which can be characterized for an indication of a urease producing bacteria. Certain embodiments described herein allow the use of these methods, combined with the detection of urease producing bacteria in less than 2-6 hours.

Aspects of the present disclosure include the use of a media that contains urea, and characterizes the microorganism for its ability to hydrolyze urea. The methods comprises the steps of i) placing bacterial organisms in a solution comprising urea and a pH indicator; and ii) examining for the production of color; where the ability to hydrolyze urea results in a pH increase. Phenol red can be used as a pH indicator and which provides for the detection of the presence of bacteria. In certain instances, the difference is that the ability to hydrolyze urea results in a pH increase, whereas the normal metabolic activity of bacteria results in a pH decrease. Certain embodiments described herein allow the use of these methods, combined with the detection of bacteria via the ability to hydrolyze urea in less than 2-6 hours.

Aspects of the present disclosure include the use of certain chromogens that changes color due to the formation of certain precipitates upon the presence of beta-galactosidase, which is produced by E. coli. In certain embodiments, chromogens of interest include those described in U.S. Pat. No. 7,807,439 (and related publications, such as J. Clin Microbiol. 2000; 38(4):1587-91), the disclosures of which are herein incorporated by reference. These disclosures involve the use of these chromogens (sometimes in combination) with agar as part of a plating media; the test sample is plated on these plates and bacterial colonies are grown overnight from the test colonies. Depending on the color of the colonies, the specific characterization of the bacteria can be discerned. Some of the specific embodiments described here allows this characterization in a faster timescale.

Aspects of the present disclosure includes methods and systems that combine a hydrophobic ligand-albumin complex, delivers this complex to the surface of the microorganism, and relies on certain chemical reactions between certain byproducts of the microorganism's metabolic activity with the hydrophobic ligand to create a product with a certain UV-Vis optical signature.

Methods according to certain embodiments include: (1) creating a first solution of albumin with the ligand that is weakly soluble in water, and in certain instances allowing sufficient time for the ligand to partition to albumin; (2) contacting a test sample that may contain an unknown bacteria with the first solution, to create a second solution; (3) determining (e.g., monitoring) the visible spectra from the second solution for a period of time; (4) detecting changes in the visible spectra; and (5) comparing the changes in the visible spectra to preset references to determine if the changes signify the presence of bacteria, or it's characterization.

Methods according to certain embodiments include: (1) creating a first solution of a ligand; (2) contacting a test sample that may contain an unknown bacteria to the first solution, to create a second solution; (3) determining (e.g., monitoring) the visible spectra from the second solution for a period of time; (4) detecting changes in the visible spectra (e.g., over a predetermined period of time); and (5) comparing the changes in the visible spectra to preset references to determine if the changes signify the presence of bacteria, or its characterization.

Methods according to certain embodiments to detect changes in the visible spectra include collecting a series of visible spectra from the solution over a period of time. The objective in certain embodiments is to discern changes in both the Rayleigh scattering contribution, and in specific changes in absorption peaks. Since most visible absorption peaks have a bandwidth of about 20 nm, and the Rayleigh contribution is spread over >100 nm, with a power law relationship between absorbance and wavelength (with an exponent of either −2, −3 or −4), the visible spectrum is typically collected with a spectral resolution <20 nm; for instance, with a resolution of about 7 nm.

Methods according to certain embodiments include: (1) methods wherein the first solution contains phenol red as the weakly soluble ligand (or any other compound whose color output is sensitive to its charge state), and the first solution includes a media that allows bacteria metabolism; (2) the specific absorption peak being monitored is at 560 nm, associated with phenol red. This peak decreases in magnitude due to acid production; (3) characterizing the rate of change of the 560 nm absorbance peak via the methods described herein, and comparing this rate against preset thresholds to signify bacteria presence; (4) Alternatively, the rate of change of the 560 nm absorbance peak can be characterized (also by the methods described herein) from multiple samples that combine the first solution with the test sample, and compare the rate of change of the 560 nm absorbance peak from those samples and with other samples that combine the first solution with a control sample. (5) Alternatively, there can be consideration of the 440 nm absorbance peak of phenol red since this peak increases in magnitude over time.

Methods according to certain embodiments include (1) the methods described herein and (2) incorporates various target antibiotics, at various concentrations to multiple second solutions and wherein (3) the rate of change of the 560 nm absorbance peak is plotted against antibiotic concentration and wherein (4) the plot of step (3) is used to determine the minimum antibiotic concentration at which the rate of change of the 560 nm absorbance peak is 0. This concentration signifies the minimum inhibitory concentration.

Methods according to certain embodiments includes the (1) the methods described herein wherein (2) the first solution includes certain Chromogenic agents that change color upon reaction with certain metabolites produced by specific bacteria, and wherein the methods described herein are used to monitor changes in the color spectrum, and wherein (3) the total integrated color spectrum is monitored over time, and a significant increase in this integrated color spectrum indicates the presence of the corresponding bacteria.

Systems for practicing the subject methods include a absorption spectroscopy monitor that can be controlled by a microcomputer, having software on the microcomputer that can implement the methods described above. In some instances, the absorbance measurement is made on a 96 well plate reader, wherein individual wells of the 96 well plate array implement specific embodiments described above. In some instances, the 96 well plates include multiple chromogen media, and multiple candidate antimicrobials.

### BRIEF DESCRIPTION OF THE DRAWINGS

FIG. 1 shows a block diagram of the method of separating an absorption spectrum into a Rayleigh scattering contribution and an absorption contribution according to certain embodiments.

FIG. 2 illustrates the first spectrum collected in a series (the “Reference” spectrum), another spectrum collected after some period of time (the “Spectrum”), and the change or difference spectrum (“Spectrum-Reference”).

FIG. 3 illustrates the difference spectrum (“Spectrum-Reference”) of FIG. 2, along with the first iteration of the power law fit with exponent −2 (the “Rayleigh fit”), and the residual, which is also the first iteration of the “Color Spectrum”. In the first iteration described here, all points are given equal weights in the fitting process. In the Color spectrum, all data points which are greater than 0 are marked with a + sign, and the weights associated with these data points are set to 0 in the iteration of FIG. 4.

FIG. 4 illustrates the difference spectrum (“Spectrum-Reference”) of FIG. 2, along with the second iteration of the power law fit with exponent −2 (the “Rayleigh fit”), and the residual, which is also the second iteration of the “Color Spectrum”. In this second iteration, the data points are given weights as per the residuals of FIG. 3. In the Color spectrum, all data points which are greater than 0 are marked with a + sign, and the weights associated with these data points are set to 0 in the iteration of FIG. 5.

FIG. 5 illustrates the difference spectrum (“Spectrum-Reference”) of FIG. 4, along with the third iteration of the power law fit with exponent −2 (the “Rayleigh fit”), and the residual, which is also the second iteration of the “Color Spectrum”. In this third iteration, the datapoints are given weights as per the residuals of FIG. 4.

FIG. 6 illustrates the color spectrum estimated as the residuals after the nth iteration, along with the peak height at 434 nm. As can be seen from the figure, the fitting process converges to a solution after 3 iteration.

FIG. 7 illustrates the detection of bacteria presence in a test sample. To 0.2 mL of a reagent that contains phenol red and a suitable media, we add 0.05 mL of a test sample that contains 1000 CFU/mL S. aureus. 3 samples were prepared, and another 3 control samples. The phenol red concentration in the reagent is adjusted to provide for a phenol red absorbance peak at 560 nm of about 2 (so as to ensure a reasonable signal to noise ratio on the optical absorbance, while being well below the saturation point of 4). Top Left: The height of the phenol red peak at 560 nm for the infected and control samples, and the ratio between these two, as a function of time; where all 6 samples are incubated at 37° C. and the visible absorbance is recorded once every 7 minutes. Note that the control sample also shows a decrease after about 350 minutes of incubation˜this is likely due to a low level contaminant in the control sample. Top Right: A close-up of the data for the first 100 minutes. There is an initial decrease in peak height for both infected and control samples˜this is likely due to the temperature changes, or due to exposure of the sample to the visible light itself. Accordingly, the decrease in peak height for the infected sample cannot be used to characterize bacteria presence; however, the ratio of the absorbance peak heights for infected and control samples accurately calibrates out these thermal/optical effects. Accordingly, a significant decrease in this quantity denotes the presence of bacteria at a concentration that is significantly above that of the background contamination. In this example, bacteria presence at 1000 CFU/mL can be determined at 63 minutes of testing.

FIG. 8A shows time variation of 560 nm phenol red peak height, as a function of pathogen concentration in the test sample. Each sample comprises 125 uL of 1× TSB, 50 uL of a phenol red solution (where the phenol red concentration is adjusted to provide for a phenol red absorbance peaks of about 2 at both 560 nm and 440 nm), and 50 uL of a test sample wherein the bacteria (E. coli 25933 in this example) concentration is varied between 0 and 108 CFU/mL.

FIG. 8B shows ratio of the absorbance peak from the test sample and that from the control sample, plotted as the reduction from the starting value.

FIG. 8C shows the time at which the ratio (middle chart) reaches 10% reduction, as a function of pathogen concentration in the test sample for E. coli and 2 other test organisms.

FIG. 9 shows an illustration of the methods used to detect urease enzyme production. Top left: Absorption spectrum for a sample that includes the 0.2 mL of a phenol red-urea broth and a Urease producing bacteria (Proteus Mirabilis in this example, added 0.05 mL of the bacteria stock at 1000 CFU/mL). The “Reference” refers to the first absorption spectrum (collected at t=0), and “Spectrum” refers to the absorption spectrum at about 4 hours. The spectra are dominated by phenol red peaks at 560 nm and 440 nm. The “change” refers to the difference in absorption spectrum at 4 hours minus the reference. Top Right: The “Change” is dominated by a Rayleigh contribution that scales as the inverse 2nd power of wavelength, and a color contribution. These contributions are separated out using the methods herein. Bottom Left: Color spectrum of identical 0.2 mL phenol red-urea broth samples that includes 0.05 mL of the test bacteria solution and a 0.05 mL control solution. Bottom Right: The average color spectrum between 500 and 600 nm for the control and bacteria samples, and the difference between the two. The difference grows exponentially over time. The presence of urease producing bacteria is flagged with the estimated exponential growth damping parameter (of the exponential fit to the difference) is greater than 0 and also greater than the confidence interval around the fitted value. In this example, urease production is flagged at 140 minutes; the phenol red markers that indicate bacteria presence (and is described FIG. 7) are flagged at 110 and 140 minutes.

FIG. 10 shows an illustration of the methods used to detect S. aureus using a commercially available mannitol-salt-phenol red (MSP) medium and the method herein. Top left: Heights of the phenol red peak, as a function of time (and normalized to the value at 20 minutes), for a sample with 150 μl of the MSP medium and 50 μl of a test solution formulated in 1× buffer with the concentrations of S. aureus indicated in the legend. Right: Same data as the chart on the left, with individual traces normalized by the output from the control sample. Bottom left: Overall Rate of change (measured over 4 hours) of the 560 nm phenol red absorbance, plotted as a function of pathogen concentration, for S. aureus and 3 other organisms. Bottom right: Final rate of change (measured over the last 10 minutes in a 4 hour measurement) of the phenol red 560 nm absorbance. S. aureus presence is indicated when either the overall rate of change (bottom left) or final rate of change (bottom right) is in the diagnostic band (shaded yellow region).

FIG. 11 shows an illustration of the methods used to characterize the minimum inhibitory concentration. This example characterizes the response of E. coli 25922 to Gentamicin. Top: The curves illustrate the response of a test solution that includes 1000 CFU/mL E. coli, the Phenol-Red based reagent described in herein, and Gentamicin GM present at concentrations that are depicted in the legend. The Y axis depicts the height of the phenol red peak at 560 nm, normalized to the starting value. Bottom: shows the normalized peak height at 300 minutes, plotted as a function of GM concentration.

FIG. 12 shows an illustration of the methods used to characterize the minimum inhibitory concentration. This example characterizes the response of E. coli 25922 to Gentamicin. Top: The curves illustrate the response of a test solution that includes 1000 CFU/mL E. coli, the Phenol-Red based reagent described herein, and Gentamicin GM present at concentrations that are depicted in the legend. The Y axis depicts the height of the phenol red peak at 560 nm, normalized to the starting value. Bottom: The normalized peak height at 300 minutes, plotted as a function of GM concentration.

FIG. 13: Doubling time (time required for pathogen concentration to double) for S. aureus ATCC 29213 versus antibiotic concentration for 5 antibiotics.

FIG. 14A: Variation of MIC estimated with CLSI M100 serial dilution methods, but with the pathogen concentration varying as shown on the X axis (instead of the 0.5 McFarland specified in the CLSI M100 method). FIG. 14B: Slope of the linear fits of the traces of MIC vs concentration, plotted against the observed MIC values.

FIG. 15: Variation of the time required for pigment detection versus antibiotic concentration for tetracycline plotted against antibiotic concentration. The pathogen concentration is estimated at 8.8×106 CFU/mL. For antibiotic concentrations of 2 μg/mL, the time to detection is increased by a factor of >2 compared to the baseline value of about 150 minutes for this pathogen concentration. We set this 2× increase as the criterion for MIC˜other criterion can also be used, but will require a different set of corrections for pathogen concentration. With the MIC at the test pathogen concentration, we apply the correction described above, and find that the rapid test MIC correlates with the CLSI M100 MIC, as depicted in FIG. 23.

FIG. 16: Color spectra for the ChromUTI Agar (150 μl) incubated with a suspension of test bacteria (50 μl of ATCC strains of various pathogenic bacteria at 1000 CFU/mL, as indicated in the legend). The bacteria can be recognized on the basis of their color spectra to varying degrees. Some of the reagents are responsive to the presence of certain types of bacteria in the sample. For instance, the Staph Selective Agar provides for color changes that are observed for both S. aureus and S. epidermidis, thus color changes in this media signifies the presence of one of these bacteria in the test sample. Likewise, the Bile Esculin Azide broth provides for a color change (visually presenting as black, on the color spectrum, the absorbance starts to rise to >2 for all wavelengths starting with the 400 nm, and then 500 and 600 nm) for Enterococcus (but not S. agalactiae), K. pneumonia and for all tested Candida organisms (C. albicans, C. glabrata, C. tropical and C. krusei). Thus the formation of a black color on the Bile Esculin Azide broth signifies the presence of one of these organisms. By extension, a black coloration on the Bile Esculin Azide broth, along with a positive on the Staph Selective Agar signifies a polymicrobial sample.

FIG. 17 Left: Measured UV-Vis absorption spectrum from a sample that contains the CromUTI Agar (HiMedia Labs M1353; formulated as per vendors direction and poured 150 μl into one well of a 96 well plate; the HiMedia Lab Strep Selective Agar also works) and an inoculum of S. aureus ATCC 29213 at (50 μL of a suspension at 104 CFU/mL. The spectra is measured with a 96 well plate reader (Versa Max from Molecular Devices, with the plate incubated at 37 C) 10 hours after addition of the test sample. The spectrum is collected with a 3 nm spectral resolution between 390 and 840 nm, using a LabView customized software running on a laptop computer that controls data acquisition and does the analysis. Right: The “color” spectra after removal of the Rayleigh contribution using the methods outlined below.

FIG. 18: Left variation of detection time (via the algorithms described in FIG. 17) versus S. aureus ATCC 29213 concentration. Right: Same variation, but for wild type strains of S. aureus.

FIG. 19: Variation of MIC estimated using the CLSI M100 methods, but with a non-standard concentration of S. aureus ATCC 29213 vs the concentration of S. aureus for 5 different antibiotics.

FIG. 20: Variation of the slope of the traces in FIG. 19 vs the magnitude of the estimated MIC at a concentration of log (CFU/mL)=6.

FIG. 21A: Variation of Rayleigh scattering with time for 7 samples containing Cation Adjusted Mueller Hinton Broth (CAMHB), an unknown concentration of a test bacteria (which was identified as S. aureus due to pigment production), and loaded onto 96 well plates at 7 concentrations of the candidate antibiotic Vancomycin, with concentrations starting at 1.25 μg/mL (for Cell 7) and decreasing in steps of 2× for each well down to Cell 1. The data is collected for 8 hours. FIG. 21B: Variation of the fitting parameter a, in the fit equation y=a/[1+exp(−(t−b)/c)], with a non-linear best fit routine is applied to fit this equation to the data depicted on the left. The fitting parameter is plotted against cell#. From this variation, it is estimated that Rayleigh growth becomes negligible at an extrapolated cell # of 4.8, which corresponds to a Vancomycin concentration of 0.19 μg/mL.

FIG. 22A: Measured UV-Vis absorption spectrum from a sample that contains the GBS medium (HiMedia Labs M1073; formulated as per vendors direction and poured 150 μl into one well of a 96 well plate) and an inoculum of S. agalactiae ATCC 27956 at (50 μL of a suspension at 103 CFU/mL. The spectra is measured with a 96 well plate reader (Versa Max from Molecular Devices, with the plate incubated at 37 C) 18 hours after addition of the test sample. The spectrum is collected with a 3 nm spectral resolution between 390 and 840 nm, using a LabView customized software running on a laptop computer that controls data acquisition and does the analysis. FIG. 22B: The “color” spectra after removal of the Rayleigh contribution using the methods outlined below for the measured spectra at 10, 15 and 25 hours after the addition of the S. agalactiae bacteria to the GBS medium. FIG. 22C: The height of the absorbance peak at 525 nm in the “color” spectrum (ie, after subtracting the Rayleigh contribution), measured over the baseline absorbance in the color spectrum (ie, the average of absorbances at 550 and 500 nm).

FIG. 23: Comparison of the described Rapid Test (results on Y axis) with the CLSI M100 disk diffusion methods (results on X axis) for 6 samples. The points marked Res and Sus are the CLSI M100 breakpoints for the serial dilution plotted against the breakpoints for the disk diffusion approach. Thus, concordance of rapid tests with CLSI methods is indicated by the data points falling in either the red (for resistant strains) or blue (for susceptible strains) squares. The solid orange line represents the best power law fit for all observed data points. Perfect concordance would be indicated by the solid orange line overlapping the blue line joining, and R\hat{ }2 values of 1. FIG. 23A: Rapid Test MIC Values after correction for pathogen concentration. FIG. 23B: Rapid Test MIC Values before pathogen concentration. Prior to the correction for pathogen concentration, the rapid test MIC values appear to have a systematic difference from the CLSI values, as evident from the distance between the orange and blue lines. Similar results were obtained for the other 11 antibiotics tested.

FIG. 24: Visual depiction of growth on ChromCandida Agar (FIG. 24A) and Bile Esculin Azide Agar (FIG. 24B) after 12 hours of incubation at 25 C with Mucor racemosus ATCC® 42647.

### DETAILED DESCRIPTION

Provided are methods of separating an absorption spectrum into a Rayleigh scattering contribution and an absorption contribution. By performing such separations and removing the influence of Rayleigh scattering, the absorption of a sample can be more accurately measured. Provided are additional methods that involve separating an absorption spectrum into a Rayleigh scattering contribution and an absorption contribution: assessing whether or not a microorganism is present in a biological fluid, assessing the effect of a pharmaceutical drug on a microorganism, and treating a subject suspected of having an infection. Provided are systems and non-transitory computer readable storage media for separating an absorption spectrum into a Rayleigh scattering and an absorption contribution.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a droplet” includes a plurality of such droplets and reference to “the discrete entity” includes reference to one or more discrete entities, and so forth.

It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

“Rayleigh scattering” refers to any method whereby light incident on a sample at a fixed wavelength is scattered at the same wavelengths via a predominantly elastic process by particles that are much smaller than the wavelength of light. For light frequencies well below the resonance frequency of the scattering particle (normal dispersion regime), the amount of scattering is inversely proportional to the fourth power of the wavelength for spherical particles. Depending on the shape of the particle, the scaling may vary between inverse 2nd power to the inverse 4th power of the wavelength.

### Methods

### Separating an Absorption Spectrum Into a Rayleigh Scattering Contribution and an Absorption Contribution

As described above, an experimentally measured absorption spectrum includes not only the absorption spectrum of an indicator, but also a Rayleigh scattering component. Thus, it is advantageous to separate these two components.

Aspects of the present disclosure include methods and systems for separating out changes in the background Rayleigh scattering from changes in specific absorption peaks. These methods are critical because the observed absorption peak is due to both the Rayleigh contribution, and specific absorption contributions; and since the Rayleigh contribution can change over time due to various reasons, absent an accurate estimation of the Rayleigh contribution, the specific absorption contribution can be estimated incorrectly. For instance, the Rayleigh contribution can change due to the presence of microbubbles in the liquid sample, and wherein the microbubbles migrate, merge, or dissipate out of the liquid. Absent robust methods to separate the Rayleigh contribution, the absorption contribution can be estimated incorrectly, thereby introducing an error (or uncertainty) in the measurement.

The method of separating an absorption spectrum into a Rayleigh scattering contribution and an absorption contribution includes the steps of:
(i) measuring an absorption spectrum
(ii) generating a fit spectrum by fitting the absorption spectrum to a power function
(iii) generating a difference spectrum by subtracting the fit spectrum from the absorption spectrum
(iv) generating an adjusted spectrum by selecting
   points from the absorption spectrum for wavelengths wherein the difference spectrum is less than or equal to zero
   points from the fit spectrum for wavelengths wherein the difference spectrum is greater than zero
(v) repeating steps (ii)-(iv) zero or more times, wherein the most recent adjusted spectrum is used in place of the absorption spectrum if the steps are repeated, wherein the final adjusted spectrum is the Rayleigh scattering contribution
(vi) generating the absorption contribution by subtracting the Rayleigh scattering contribution from the absorption spectrum

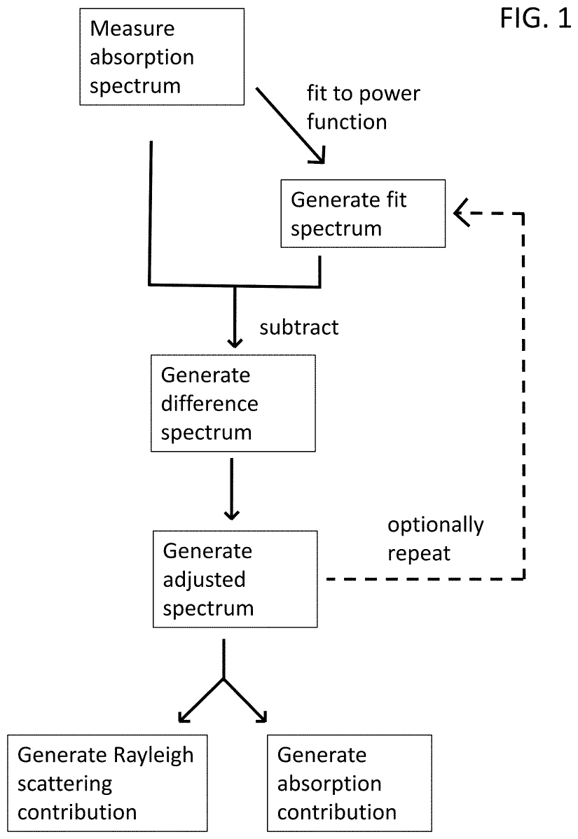

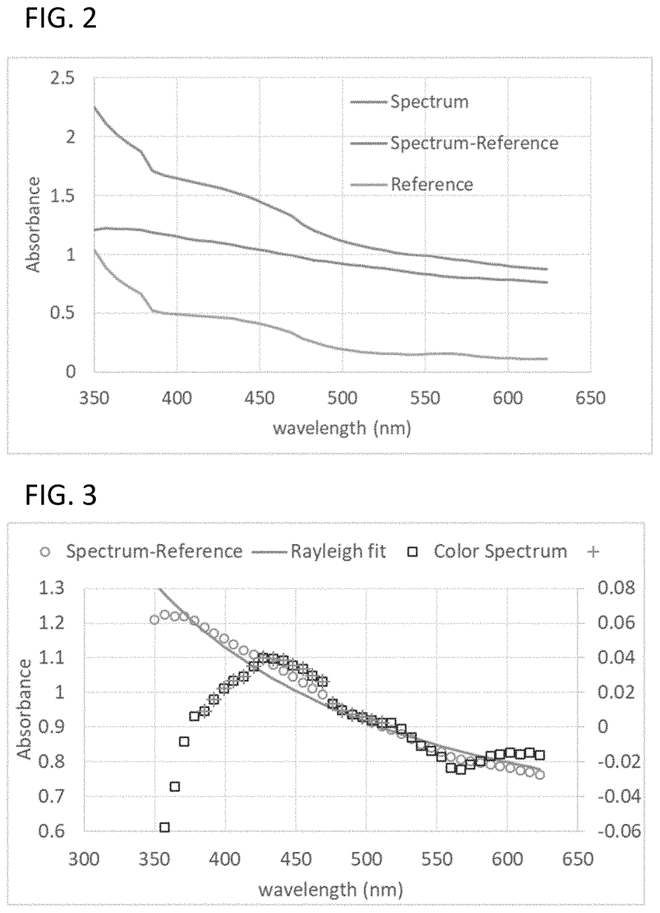

FIG. 1 shows a block diagram of the steps discussed above. FIG. 2-FIG. 5 show an exemplary embodiment of the method. The exemplary embodiment is discussed first, followed by a discussion of the general method.

In FIG. 2, the bottom curve is a reference spectrum that was measured before the experiment. The top curve is the spectrum measured during the experiment. The middle curve is the result of subtracting the reference from the spectrum measured during the experiment. As such, in this embodiment the optional step of correcting for a reference spectrum is performed. As such, the “absorption spectrum” measured in step (i) above is the middle spectrum of FIG. 2.

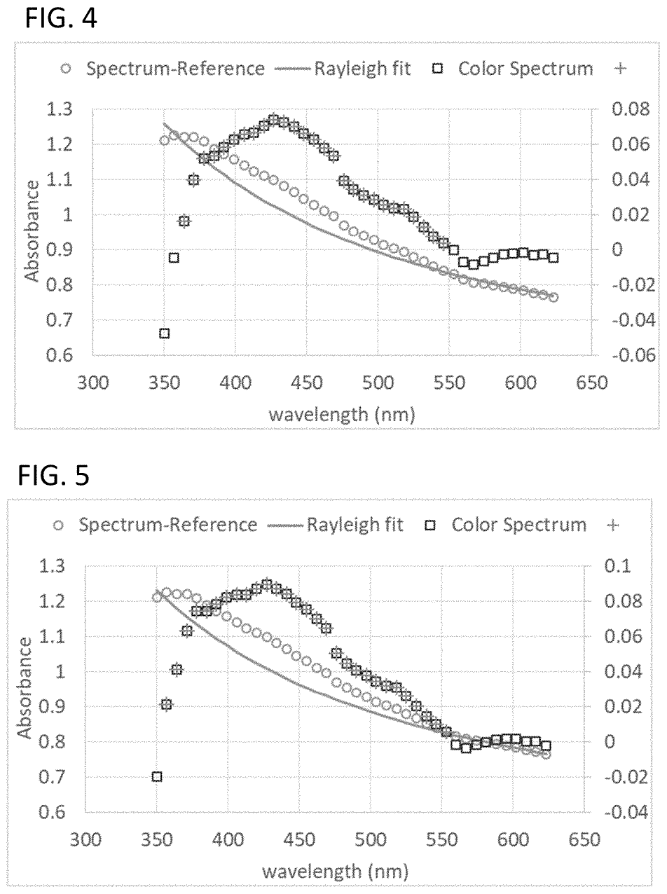

In FIG. 3, the middle spectrum of FIG. 2 is converted to a series of blue circles (representing individual data points) beginning at 350 nm and 1.2 absorbance. This absorption spectrum is fit to a power function, generating the “fit spectrum” that is the solid line labeled as “Rayleigh fit”. FIG. 3 also shows the step of generating a difference spectrum. By subtracting the fit spectrum from the absorption spectrum, the difference spectrum is generated, which is shown as red squares beginning at 350 nm and −0.06 (axis on right side of the figure). Also shown in FIG. 3 is a “plus” (+) mark wherein the difference spectrum is positive.

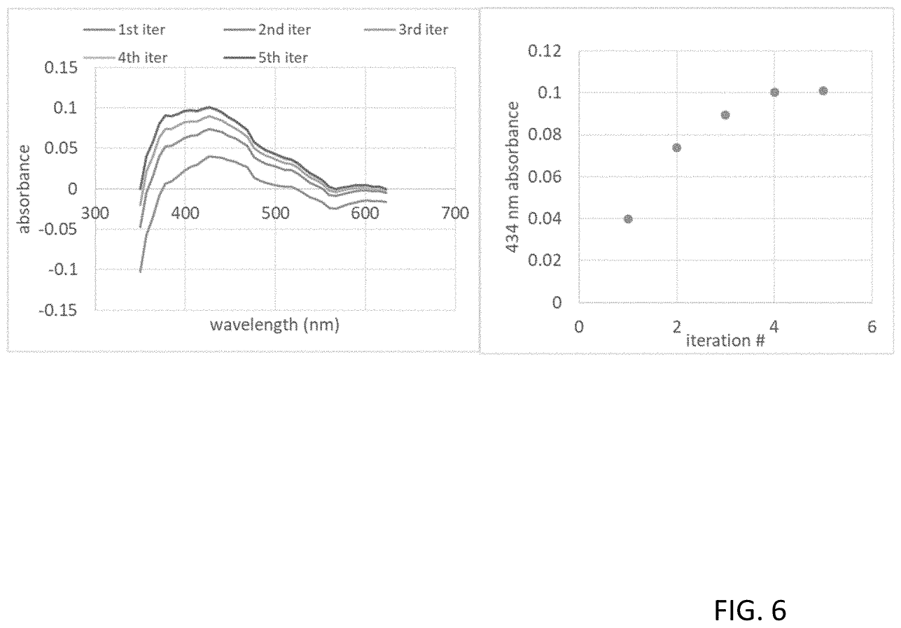

In FIG. 4, the blue circles represent the first adjusted spectrum, which was generated by selecting points from the absorption spectrum in FIG. 3 and based on the difference spectrum of FIG. 3. The points were selected according to the algorithm discussed above in step (iv). FIG. 4 also shows a first repetition of steps (ii)-(iv). Namely, the first adjusted spectrum is fit to a power function, generating a fit function shown as the solid line and labeled as “Rayleigh fit”. A difference spectrum is then generated, which is shown as red squares and labeled as “color spectrum”. Plus (+) marks are shown where the difference spectrum is positive.

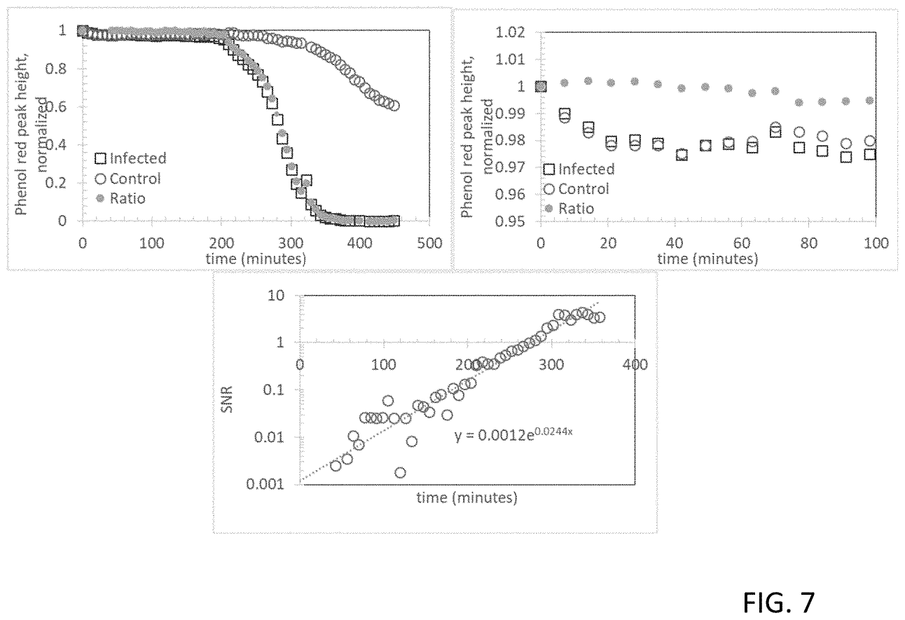

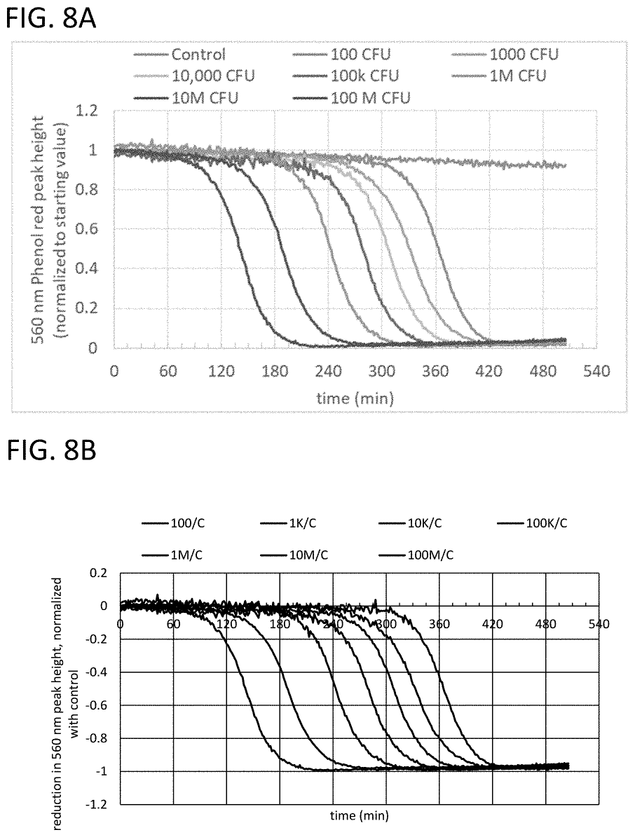

In FIG. 5, the above sequence is repeated once again. As this is the last repetition, the final adjusted spectrum can be considered as the Rayleigh contribution. The absorption contribution is the original spectrum minus the Rayleigh contribution.

By “generating a fit spectrum” is meant that a mathematical regression is performed on the data points collected as part of the absorption spectrum. In the case of the present method, the fit spectrum generated involves fitting the absorption spectrum to a power function. For instance, if wavelength is graphed on the x-axis and absorbance is graphed on the y-axis, the power function being fit can take the form of y=a·x\hat{ }n+C, wherein “a” and “C” are constants and “n” is the power to which the function is raised. Since Rayleigh scattering varies with inverse values of wavelength, the value of “n” will be negative. For example, n can be −2, −3, or −4. In some cases, “n” is a whole number. In other cases, “n” is not a whole number.

After a fit spectrum is generated, the next step of the method is generating a difference spectrum by subtracting the fit spectrum from the absorption spectrum. This step can optionally involve actually plotting or graphing the resulting difference spectrum, but such is not required. As an example, if the absorbance in the absorbance spectrum is 0.90 at 400 nm and the absorbance in the fit spectrum is 0.85 at 400 nm, then the value of the difference spectrum will be 0.05 at 400 nm. The values of the difference spectrum can be either positive or negative. As another example, if at 600 nm that absorbance value was 0.40 and the fit value was 0.55, then the difference spectrum at 600 nm would be −0.15.

Next, the adjusted spectrum is generated by selecting absorbance values from either the fit spectrum or the absorption spectrum, based on whether the difference spectrum is positive or negative. Continuing with the above example, at 400 nm the difference spectrum had a positive value of 0.05. Therefore, the algorithm dictates that the fit value of 0.85 is selected for the adjusted spectrum at 400 nm. In contrast, since the value of the difference spectrum at 600 nm is negative at −0.15, the algorithm dictates that the absorption value of 0.40 is selected for the adjusted spectrum at 600 nm.

After generating the adjusted spectrum, step (v) involves the optional repetition of steps (ii) through (iv) zero or more times. Thus, in some cases, the steps are not repeated, and the method continues to step (vi). In other cases, the steps are repeated, in which case the most recent adjusted spectrum is used in place of the experimentally-measured, original absorption spectrum. This repetition can be repeated any suitable number of times, such as 0, 1, 2, 3, 4, 5, 10, or 15 or more. In some cases, the step is repeated 1 or more times, such as 2 or more times.

The final adjusted spectrum generated in step (v) is the approximation of the Rayleigh scattering contribution. Due to the power law shape of the function, it approximates the natural behavior of Rayleigh scattering. In order to obtain the absorption contribution, the Rayleigh scattering contribution is subtracted from the original, experimentally measured absorption spectrum.

The absorption spectrum can be measured using any suitable instrument, e.g., one that measures some or all of the UV-Visible electromagnetic range. In some cases, the absorption spectrum can include a wavelength within the range of 250 nm to 800 nm, such as 350 nm to 650 nm.

The power function typically has an order ranging from −2 to −4. In some cases, the power function has a whole number order, e.g., −2, −3, or −4. In some cases, the power function has a non-whole number order, e.g., between −2 and −3 or between −3 and −4. In cases wherein some steps of the method are repeated, as described above, the order of the power function can be either the same or different for each repetition. In some cases, the order begins at a whole number, but becomes a non-whole number in subsequent repetitions.

Any suitable indicator of microbial presence can be employed. In some cases, the indicator is a pH sensitive dye that changes its absorption spectrum in response to a change in pH, e.g., phenol red.

### Detection of Microorganisms

Provided are methods for assessing whether or not microorganisms are present in a biological fluid. In some cases the method includes:
(i) for both the biological fluid and a sterile fluid:
   (a) contact the fluid with a detection component that changes its optical absorbance in response to a metabolic product of the microorganisms
   (b) measure a reference optical absorption spectrum at an initial time
   (c) measure a plurality of subsequent optical absorption spectrums at subsequent times
   (d) generating a plurality of Rayleigh-corrected spectrums by correcting the subsequent optical absorption spectrums for contributions from Rayleigh scattering
   (e) creating a two-dimensional plot using the Rayleigh-corrected spectrums, wherein one axis of the plot is time since the reference spectrum, wherein one axis of the plot is the change in absorbance at a particular wavelength or in a particular wavelength range since the reference spectrum
(ii) determining whether or not bacteria are present in the biological fluid by comparing the two-dimensional plot of the biological fluid to the two-dimensional plot of the sterile fluid
(iii) reporting whether or not bacteria was determined to be present in the biological fluid

The microorganism can be a bacteria, a virus, an amoeba, or a fungi. In some cases, the power function has an order of −2, −3, or −4. The order can be the same or different between each of the optional repetitions. In some cases, the steps are repeated 1 time, 2 times, 3 times, or 4 or more times. In some cases the determining comprises comparing the rate of change of the two-dimensional plot of the biological fluid to a present threshold. In some cases, the detection component changes its optical absorbance in response to a change in pH. In some cases the detection component changes its optical absorbance in response to an enzyme produced by a bacteria. In some cases, the enzyme is a urease enzyme and the biological fluid is contacted with urea before the other steps of the method. In some cases the detection component is blood or urine.

### Quantification of Microorganisms

Provided are methods for quantifying the amount of a microorganism present in a biological fluid. In some cases, the method includes:
(i) for both the biological fluid and a sterile fluid:
   (a) contact the fluid with a detection component that changes its optical absorbance in response to a metabolic product of the microorganisms
   (b) measure a reference optical absorption spectrum at an initial time
   (c) measure a plurality of subsequent optical absorption spectrums at subsequent times
   (d) generating a plurality of Rayleigh-corrected spectrums by correcting the subsequent optical absorption spectrums for contributions from Rayleigh scattering
   (e) creating a two-dimensional plot using the Rayleigh-corrected spectrums, wherein one axis of the plot is time since the reference spectrum, wherein one axis of the plot is the change in absorbance at a particular wavelength or in a particular wavelength range since the reference spectrum
(ii) determining the amount of the microbe in the biological fluid by comparing the two-dimensional plot of the biological fluid to the two-dimensional plot of the sterile fluid
(iii) reporting the amount of the microbe determined to be present in the biological fluid

### Assessing the Effect of Pharmaceutical Drugs on Microorganisms

Provided are methods of assessing the effect of a pharmaceutical drug on a microorganism. In some cases, the method includes:
(i) for both a first fluid comprising the microorganism and the pharmaceutical drug and for a second fluid comprising the microorganism and lacking the pharmaceutical drug:
   (a) contact the fluid with a detection component that changes its optical absorbance in response to a metabolic product of the microorganisms
   (b) measure a reference optical absorption spectrum at an initial time
   (c) measure a plurality of subsequent optical absorption spectrums at subsequent times
   (d) generating a plurality of Rayleigh-corrected spectrums by correcting the subsequent optical absorption spectrums for contributions from Rayleigh scattering
   (e) creating a two-dimensional plot using the Rayleigh-corrected spectrums, wherein one axis of the plot is time since the reference spectrum, wherein one axis of the plot is the change in absorbance at a particular wavelength or in a particular wavelength range since the reference spectrum
(ii) determining the effect of the pharmaceutical drug on the microorganism by comparing the two-dimensional for the first fluid to the two-dimensional plot for the second fluid
(iii) reporting the effect of the pharmaceutical drug on the microorganism

In some cases, the microorganism is a bacteria and the pharmaceutical drug is an antibiotic. In some cases, the method further comprises performing steps (i)-(iii) for a third fluid comprising the microorganism and the pharmaceutical drug at a concentration different than the pharmaceutical drug concentration in the first fluid.

### Treating a Subject Suspected of Having an Infection

Provided are methods of treating a subject suspected of having an infection. In such cases, the method comprises performing or having performed a method of determining whether a microbe is present in a biological fluid and quantifying a microbe present in a biological fluid. In some cases, the method further comprises administering a pharmaceutical drug to the subject based on the determination, e.g., an antibiotic.

### Systems

### Systems for Separating an Absorption Spectrum Into a Rayleigh Scattering Contribution and an Absorption Contribution

Provided are systems for separating an absorption spectrum into a Rayleigh scattering contribution and an absorption contribution, comprising:
a light source;
a detector; and
a processor comprising memory operably coupled to the processor wherein the memory comprises instructions stored thereon, which when executed by the processor, cause the processor to:
   irradiate a sample with the light source;
   record an absorption spectrum with the detector; and
   separate the absorption spectrum into a Rayleigh scattering contribution and an absorption contribution

In some cases, the separating comprises the steps of the methods described above. In some cases, the instructions are configured for irradiation, recordation, and separation for a plurality of samples. The plurality of samples can be 2 or more, such as 5 or more, 10 or more, 25 or more, 50 or more, or 100 or more.

### Systems for Absorbance Spectroscopy

Aspects of the present disclosure also include sub-systems for absorbance spectroscopy and sub-systems for computing absorbance peak heights from an absorbance spectrum, sub-systems for tracking absorbance peak heights over time, and sub-systems for rendering that information into pathogen ID and antimicrobial susceptibility information.

Sub-systems according to certain embodiments include a absorbance spectrometer, which includes broadband light source (such as a Tungsten Halogen bulb), monochromator that selects certain wavelengths from that broadband light source, a computer than can command the monochromator to select certain wavelengths, a collimating stage that accepts a sample cell and a detector that characterizes light intensity after it has passed through the sample cell and a processor having memory operably coupled to the processor, the memory having instructions stored thereon, which when executed by the processor, cause the system to execute the following steps: (1) select a first desired wavelength to be selected by the monochromator (2) cause the sample to be irradiated with the first desired wavelength (3) determine a first measured intensity of light at the first desired wavelength at the photodide (4) calculate the absorbance of the sample by calibrating this first measured intensity with a second measured intensity without the sample present (5) repeat the steps (1)-(4) until a spectrum of absorbance vs wavelength covering the desired wavelength region is obtained.

Sub-systems according to certain embodiments include processors with built in memory to compute the height of the absorbance peak from an absorption spectrum collected with the subsystem herein. The memory has instructions, which upon execution, causes the following steps to be executed: (1) The measured absorbance spectrum treated as a first absorbance spectrum. (2) The first absorbance spectrum is fitted with a power function (absorbance is a function of the inverse n-th power of wavelength, where n is either 2, or 3 or 4). (3) The “residuals” (i.e., the difference between the fitted absorbance spectrum of Step 1 and the measured absorbance spectrum) is computed. (4) From the residuals, a root-mean-square residual is computed. (5) For all wavelengths for which is the residual is greater than the root-mean-square residual, the absorbance value of the first absorbance spectrum is replaced by the absorbance value in the power spectrum, and a second absorbance spectrum is thus created. (6) The steps (2)-(5) are repeated for a total of 4 times. The final fitted power function is now treated as a fit to the Rayleigh absorbance spectrum. (7) The absorbance peak heights are computed as the difference of the original absorbance spectrum and the final Rayleigh absorbance spectrum of Step 6.

Sub-systems according to certain embodiments include processors with built in memory to track the height of the absorbance peak from an absorption spectrum collected with the subsystem herein and analyzed via the subsystem of herein. The memory has instructions, which upon execution, causes the following steps to be executed: (1) Initiate the measurement at the first timepoint. (2) Measure the absorbance spectrum for the sample, using the subsystem of herein (3) Analyze the absorbance spectrum to compute the absorbance peak, using the subsystem of herein. (4) Wait for a fixed duration of time. This duration is smaller than the time period of the test. For instance, the tests described here take about 2-6 hours to complete. Accordingly, the duration of this waiting period can be less than 2-6 hours; for example about 10 minutes. (5) Repeat the steps of (2)-(3) and compute the absorbance peak height at each time point.

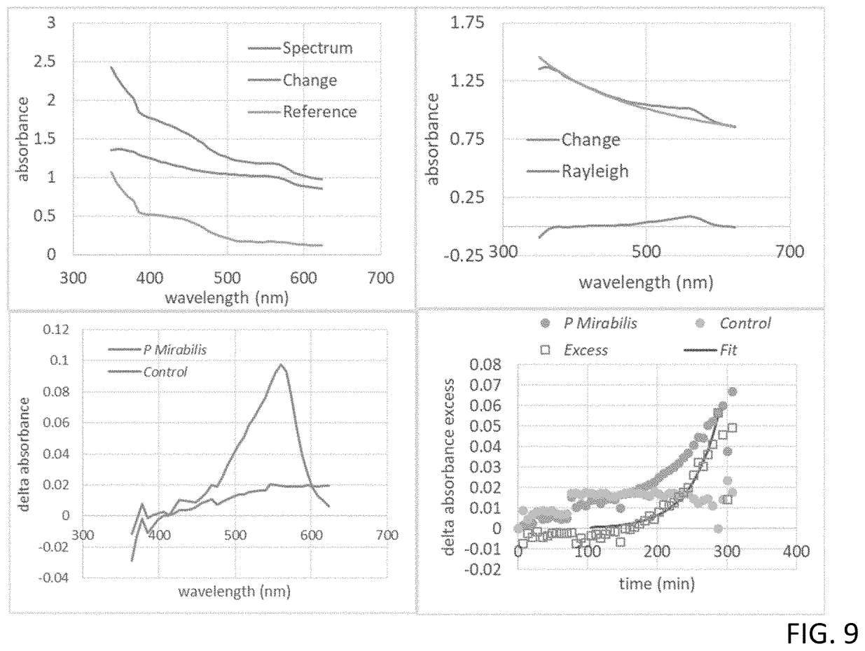

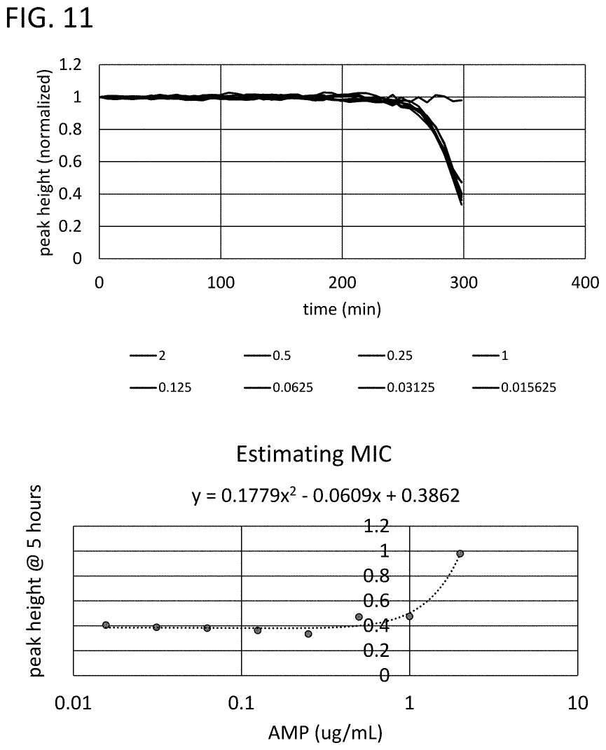

Sub-systems according to certain embodiments include processors with built in memory to characterize the antimicrobial susceptibility, and to subclassify the pathogen for ID via the subsystems that track the height of the absorbance peak of above, using absorption spectrum collected with the subsystem of herein and analyzed via the subsystem of herein. The memory has instructions, which upon execution, causes multiple steps to be executed. (1) The “sample” comprises a 96 well plate with predetermined test samples; wherein each well has a different test reagent for either antimicrobial susceptibility, or pathogen subclassification. The memory has preset information that corresponds to this preset on the 96 well plate, and causes absorbance peaks from each of those 96 wells to be read over time. It then stores the absorbance peak heights over time for each of those 96 wells. (2) From the preset wells that correspond to the reagents described in above, the memory computes the concentration of bacteria present tin the test sample, using scaling curves of FIGS. 8A-C as a guideline. Specifically, the memory computes the reduction in phenol red peak height, and then uses this reduction to read the expected bacteria concentration. (3) From the preset wells that include a reagent specific to a particular bacteria, for example, the Staphylococcus specific reagent of above, the memory computes the expected concentration of the specific bacteria using the curves of FIG. 10 as a guidelines. (4) From the preset wells that include the reagents of in above, and varying concentrations of a test antibiotic, the memory computes the minimum inhibitory concentration using the methods of above.

In these subsystems, the decision-making thresholds may vary from those dictated by master curves, ranging from 0.8 to 1.2, such as from 0.85 to 1.15, such as from 0.9 to 1.1 and including a predetermined bias to the decisions that are designed to reduce risk to the patient.

As summarized above, systems include one or more light sources, and sample chambers that can accept 96 well plates with 96 distinct samples. In embodiments, light sources of interest output light having a narrow range of wavelengths, such as a range of 25 nm or less, such as 20 nm or less, such as 15 nm or less, such as 10 nm or less, such as 5 nm or less and including 2 nm or less.

In certain embodiments, the 96 well plates are replaced by plates with other number of finite wells. In certain embodiments, a robotic arm is added to load and unload the 96 well plates. In some embodiments, the system is calibrated prior to any measurement by measuring a blank (or empty) sample chamber. This calibration curve is stored in the memory, and is subtracted from the sample measurement.

As described above, methods include irradiating a sample with a light of particular wavelength and determining the intensity of transmitted light. Systems for practicing the subject methods include one or more detectors for detecting light. Any convenient light detection protocol may be employed, including but not limited to photosensors or photodetectors such as active-pixel sensors (APSs), quadrant photodiodes, wedge detectors image sensors, charge-coupled devices (CCDs), intensified charge-coupled devices (ICCDs), light emitting diodes, photon counters, bolometers, pyroelectric detectors, photoresistors, photovoltaic cells, photodiodes, photomultiplier tubes, phototransistors, quantum dot photoconductors or photodiodes and combinations thereof, among other photodetectors. In certain embodiments, systems include one or more CCDs.

Where the subject systems include more than one photodetector, each photodetector may be the same or a combination of different types of photodetectors. For example, where the subject systems include two photodetectors, in some embodiments the first photodetector is a CCD-type device and the second photodetector is a CMOS-type device. In other embodiments, both the first and second photodetectors are CCD-type devices. In yet other embodiments, both the first and second photodetectors are CMOS-type devices. In yet other embodiments, the first photodetector is a CCD-type photodetector or CMOS-type device and the second photodetector is a photomultiplier tube. In still other embodiments, the first photodetector and the second photodetector are photomultiplier tubes.

The detector may be optically coupled to one or more optical adjustment components. For example, systems may include one or more lenses, collimators, pinholes, mirrors, beam choppers, slits, gratings, filters, light refractors, and any combinations thereof. In some embodiments, the detector is coupled to a wavelength separator, such as colored glass, bandpass filters, interference filters, dichroic mirrors, diffraction gratings, monochromators and combinations thereof. In certain embodiments, transmitted light from the sample is collected with fiber optics (e.g., fiber optics relay bundle) and is conveyed to the detector surface through the fiber optics. Any fiber optics light relay system may be employed to propagate the scattered light onto the active surface of the detector.

In embodiments, absorbance measurements are conducted at a substantially constant temperature. As such, the subject systems are configured to maintain a substantially constant temperature, such as where the temperature of the system changes by 5° C. or less, such as by 4.5° C. or less, such as by 4° C. or less, such as by 3.5° C. or less, such as by 3° C. or less, such as by 2.5° C. or less, such as by 2° C. or less, such as by 1.5° C. or less, such as 1° C. or less, such as by 0.5° C. or less, such as by 0.1° C. or less, such as by 0.05° C. or less, such as by 0.01° C. or less, such as by 0.005° C., such as by 0.001° C., such as by 0.0001° C., such as by 0.00001° C. or less and including by 0.000001° C. or less. In embodiments, the temperature of the system may be controlled by a temperature control subsystem which measures the system temperature and if necessary, controls the ambient conditions to maintain a desired system temperature. Temperature subsystems may include any convenient temperature control protocol, including, but not limited to heat sinks, fans, exhaust pumps, vents, refrigeration, coolants, heat exchanges, Peltier or resistive heating elements, among other types of temperature control protocols.

As summarized above, systems include one or more processors having memory that includes instructions stored for practicing the methods described above. In some embodiments, the memory includes instructions stored thereon.

The computer-implemented method described herein can be executed using programming that can be written in one or more of any number of computer programming languages. Such languages include, for example, Java (Sun Microsystems, Inc., Santa Clara, Calif.), Visual Basic (Microsoft Corp., Redmond, Wash.), and C++ (AT&T Corp., Bedminster, N.J.), as well as any many others.

The computer readable storage medium may be employed on one or more computer systems having a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C++, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

### Non-Transitory Media

Also provided are non-transitory computer readable storage media. Such media can be, for example, a CD-ROM, a USB drive, a floppy disk, or a hard drive. In some cases, the medium comprises instructions stored thereon for separating an absorption spectrum into a Rayleigh scattering contribution and an absorption contribution. In some cases, the instructions comprise:
(i) an algorithm for measuring an absorption spectrum
(ii) an algorithm for generating a fit spectrum by fitting the absorption spectrum to a power function
(iii) an algorithm for generating a difference spectrum by subtracting the fit spectrum from the absorption spectrum
(iv) an algorithm for generating an adjusted spectrum by selecting
   points from the absorption spectrum for wavelengths wherein the difference spectrum is less than or equal to zero
   points from the fit spectrum for wavelengths wherein the difference spectrum is greater than zero
(v) an algorithm for repeating steps (ii)-(iv) zero or more times, wherein the most recent adjusted spectrum is used in place of the absorption spectrum if the steps are repeated, wherein the final adjusted spectrum is the Rayleigh scattering contribution
(vi) an algorithm for generating the absorption contribution by subtracting the Rayleigh scattering contribution from the absorption spectrum

Notwithstanding the appended claims, the disclosure is also defined by the following clauses:
1. A method of separating an absorption spectrum into a Rayleigh scattering contribution and an absorption contribution, comprising:

(i) measuring an absorption spectrum;

(ii) generating a fit spectrum by fitting the absorption spectrum to a power function;

(iii) generating a difference spectrum by subtracting the fit spectrum from the absorption spectrum;

(iv) generating an adjusted spectrum by selecting;

points from the absorption spectrum for wavelengths wherein the difference spectrum is less than or equal to zero, and

points from the fit spectrum for wavelengths wherein the difference spectrum is greater than zero;

(v) repeating steps (ii)-(iv) zero or more times, wherein the most recent adjusted spectrum is used in place of the absorption spectrum if the steps are repeated, wherein the final adjusted spectrum is the Rayleigh scattering contribution; and

(vi) generating the absorption contribution by subtracting the Rayleigh scattering contribution from the absorption spectrum.
2. A method of assessing whether or not microorganisms are present in a biological fluid, comprising:

(i) for both the biological fluid and a sterile fluid:
(a) contact the fluid with a detection component that changes its optical absorbance in response to a metabolic product of the microorganisms;
(b) measure a reference optical absorption spectrum at an initial time;
(c) measure a plurality of subsequent optical absorption spectrums at subsequent times;
(d) generating a plurality of Rayleigh-corrected spectrums by correcting the subsequent optical absorption spectrums for contributions from Rayleigh scattering;
(e) creating a two-dimensional plot using the Rayleigh-corrected spectrums wherein one axis of the plot is time since the reference spectrum, wherein one axis of the plot is the change in absorbance at a particular wavelength or in a particular wavelength range since the reference spectrum; and

(ii) determining whether or not bacteria are present in the biological fluid by comparing the two-dimensional plot of the biological fluid to the two-dimensional plot of the sterile fluid.
3. A method of assessing whether or not a specific microorganism is present in a biological fluid, comprising:

(a) mixing the biological fluid with a reagent that is preselected to produce a specific response when the test microorganism is present:

(b) contact the biological fluid and reagent with a detection system that measures its optical absorbance at multiple wavelengths;

(c) measure a reference positive control optical absorption spectrum at an initial time using a known sample that contains the microorganism mixed with the reagent and a negative control optical absorption spectrum using a known sample that does not contain the microorganism mixed with the reagent;

(d) measure a “test” optical absorption spectrum from the biological fluid and reagent;

(d) generating a plurality of Rayleigh-corrected spectra by correcting one or more of the optical absorption spectra from the positive control, the negative control and the test sample;

(e) determining whether or not the bacteria in the positive control is present in the biological fluid by comparing the spectra from the test sample with the spectra of the positive and negative controls.
4. The method according to 3, wherein the method comprises determining if certain absorption peaks are present in the test sample by estimating if the absorption at the associated wavelength rises above a threshold.
5. The method according to 3, wherein the method comprises determining if the maximum in the absorbance vs wavelength profile for the test sample is within a preset range associated with the positive control.
6. The method according to 3, wherein the method comprises determining if the absorbance ratios at two wavelengths are within a range associated with the positive control.
7. The method according to 3, wherein the method comprises determining if the absorbance at predetermined wavelengths exceeds a present threshold.
8. The method according to 7, wherein the predetermined wavelengths are not associated with an absorption peak.
9. The method according to 3, wherein the method comprises determining if the time dependent absorbance at particular wavelengths exceeds a preset threshold for a period of time.
10. The method according to 9, wherein the determining that the dependent absorbance at particular wavelengths exceeds a preset threshold for a period of time and reverts to values below the preset threshold.
11. The method according to 3, wherein the method comprises reporting whether or not bacteria was determined to be present in the biological fluid.
12. The method of any one of 3-11, wherein correcting the subsequent optical absorption spectrums for contributions from Rayleigh scattering comprises for each of the plurality of subsequent optical absorption spectrums:

(a) generating a change spectrum by subtracting the reference optical absorption spectrum from the subsequent optical absorption spectrum;

(b) generating a fit spectrum by fitting the change spectrum to a power function;

(c) generating a difference spectrum by subtracting the fit spectrum from the change spectrum;

(d) generating an adjusted spectrum by selecting:
points from the change spectrum for wavelengths wherein the difference spectrum is less than or equal to zero, and
points from the fit spectrum for wavelengths wherein the difference spectrum is greater than zero;

(e) repeating steps (a)-(c) zero or more times, wherein the most recent adjusted spectrum is used in place of the change spectrum if the steps are repeated, wherein the final adjusted spectrum is a Rayleigh profile; and

(f) generating a Rayleigh-corrected spectrum by subtracting the Rayleigh profile from the subsequent optical absorption spectrum.
13. The method of any one of 2-12, wherein the microorganisms are bacteria.
14. The method of any one of 2-13, wherein the power function has an order of −2, −3, or −4, wherein the order can be same or different between each of the optional repetitions.
15. The method of any one of 12-14, wherein steps (a)-(c) are repeated 1 time, 2 times, or 3 times.
16. The method of any one of 2-15, wherein the determining comprises comparing the rate of change in the two-dimensional plot of the biological fluid to a preset threshold.
17. The method of any one of 2-16, wherein the detection component changes its optical absorbance in response a change in pH.
18. The method of any one of 2-17, wherein the detection component changes its optical absorbance in response to an enzyme produced by a bacteria.
19. The method of any one of 2-18, wherein the enzyme is a urease enzyme, wherein the contacting step further comprises contacting the fluid with urea.
20. The method of any one of 2-19, wherein the detection component comprises phenol red.
21. The method of any one of 2-20, wherein the biological fluid is blood or urine.
22. A method of assessing the effect of a pharmaceutical drug on a microorganism, comprising

(i) for both a first fluid comprising the microorganism and the pharmaceutical drug and for a second fluid comprising the microorganism and lacking the pharmaceutical drug:
(a) contact the fluid with a detection component that changes its optical absorbance in response to a metabolic product of the microorganisms;
(b) measure a reference optical absorption spectrum at an initial time;
(c) measure a plurality of subsequent optical absorption spectrums at subsequent times;
(d) generating a plurality of Rayleigh-corrected spectrums by correcting the subsequent optical absorption spectrums for contributions from Rayleigh scattering;
(e) creating a two-dimensional plot using the Rayleigh-corrected spectrums, wherein one axis of the plot is time since the reference spectrum, wherein one axis of the plot is the change in absorbance at a particular wavelength or in a particular wavelength range since the reference spectrum; and

(ii) determining the effect of the pharmaceutical drug on the microorganism by comparing the two-dimensional for the first fluid to the two-dimensional plot for the second fluid.
23. The method according to 22, wherein the method further comprises reporting the effect of the pharmaceutical drug on the microorganism.
24. A method of assessing the presence of a microorganism, the method comprising:

for a series of test samples that comprise all the test biological fluid with the unknown microorganism at the unknown concentration, a reagent media that supports microorganism growth and generates optical absorption, and a candidate antibiotic or pharmaceutical drug present at a series of concentrations that start at a high concentration above the resistant breakpoint and decreasing in factors of 2 such that the lowest concentration is below the susceptible breakpoint;

contacting the test samples with a detection component that changes its optical absorbance in response to a metabolic product of the microorganisms;

measuring a reference optical absorption spectrum at an initial time associated with a positive and a negative control, wherein the positive control include test samples comprises the microorganism present at a plurality of predetermined concentrations;

determining the time required to detect microorganism presence, and creating a master curve of time versus concentration of microorganism in the positive control;

measure a plurality of optical absorption spectrums at subsequent times from all the test samples;

generating a plurality of Rayleigh-corrected spectrums by correcting the subsequent optical absorption spectrums for contributions from Rayleigh scattering; and

determining the presence of the microorganism by comparing the test samples with positive and negative controls.
25. The method according to 24, wherein the method further comprises determining the concentration of the microorganism in the test sample by comparing the time required to determine microorganism presence with a master curve.
26. The method of assessing an effect of a pharmaceutical drug on an unknown microorganism present in a biological fluid, according to any one of 24-25, wherein the method further comprises:

creating a set of samples wherein the concentration of the candidate pharmaceutical drug varies from a high concentration above the resistant breakpoint to a low concentration below the susceptible breakpoint;

plotting the time required to determine microorganism presence versus the concentration of the pharmaceutical drug from all the known samples that differ only in the concentration of the pharmaceutical drug; and

thresholding the concentration at which microorganism concentration does not change significantly from starting values.
27. The method according to any one of 24-26, wherein the method further comprises determining a minimum inhibitory concentration (MIC) by determining the threshold concentration at which the time required for determining microorganism presence increases by a preset factor above the baseline value.
28. The method according to any one of 25-27, wherein the method further comprises correcting the MIC for a standard pathogen concentration by using the concentration of the microorganism in the test sample determined by comparing the time required to determine microorganism presence with the master curve.
29. The method according to any one of 25-27, wherein the method further comprises correcting the MIC for a standard pathogen concentration by using the concentration of the microorganism in the test sample determined by the threshold concentration at which the time required for determining microorganism presence increases by a preset factor above the baseline value.
30. The method according to any one of 25-27, wherein the method further comprises correcting the MIC for a standard pathogen concentration by a predetermined master curve of the variation of MIC with pathogen concentration vs the absolute value of the estimated MIC.
31. The method according to any one 24-30, wherein the method further comprises characterizing a resistant, susceptible or intermediate status of the microorganism by comparing it against predetermined breakpoints.
32. The method according to any one of 24-31, wherein the microorganism is bacteria and the pharmaceutical drug is an antibiotic.
32A. A method of assessing the effect of a pharmaceutical drug on a microorganism, the method comprising the following steps:
(i) for a series of test samples that comprise all the test biological fluid with the unknown microorganism at the unknown concentration, a reagent media that supports microorganism growth and also enables the production of optical absorption features, and a candidate antibiotic or pharmaceutical drug present at a series of concentrations that start at a high concentration above the resistant breakpoint (defined in the CLSI M100 handbook) and decreasing in factors of 2 such that the lowest concentration is below the susceptible breakpoint (also defined in the CLSI M100 handbook)
(ii) contact the test samples with a detection component that changes its optical absorbance in response to a metabolic product of the microorganisms;
(iii) measure a reference optical absorption spectrum at an initial time associated with positive and negative controls, wherein the positive controls include test samples prepared with the test microorganism present at multiple known concentrations.
(iv) Determining the time required to detect microorganism presence, and creating a master curve of this time versus concentration of microorganism in the positive control
(v) measure a plurality of subsequent optical absorption spectrums at subsequent times from all the test samples; generating a plurality of Rayleigh-corrected spectrums by correcting the subsequent optical absorption spectrums for contributions from Rayleigh scattering;
(vi) determining the presence of the specific microorganisms by comparing the essential features from the test samples with positive and negative controls.
(vii) determining the concentration of the specific microorganism in the test sample by comparing the time required to determine microorganism presence with the master curve of step iv.
(viii) Plotting the time required to determine microorganism presence versus the concentration of the test antibiotic (or pharmaceutical drug) from all the known samples that differ only in the concentration of the test antibiotic (or pharmaceutical drug)
(ix) Determining the MIC by looking up the threshold concentration at which the time required for determining microorganism presence increases by a preset factor above the baseline value
(x) Correcting the MIC for a standard pathogen concentration by using the concentration determined in Step vii, the MIC determined from Step ix and a pre determined master curve of the variation of MIC with pathogen concentration vs the absolute value of the estimated MIC
(xi) Characterizing the “resistant” vs “susceptible” vs “intermediate” status of the microorganism by comparing it against the breakpoints reported in the CLSI M100 handbook, or the breakpoints determined by other means if they are not listed in the CLSI handbook.
33. The method according to any one of 24-32A, wherein the microorganism is elected from the group consisting of E. coli, S. epidermidis, S. aureus, K. pneumonia, P. Mirabilis, A. baumannii, Enterococcus, S. agalactiae, Candida organisms, Mucor Organisms, wherein selected examples include:
   E. coli. Growth in the absorption peak between 540 and 570 nm in the CromUTI agar, and the green color detection in HiColiform Broth
   (ii) S. epidermidis: Distinct color development on CromUTI agar, distinct changes in Staph Selective Agar, and absence of pigment signatures associated with S. aureus
   (iii) S. aureus: Distinct color development on CromUTI agar, distinct changes in Staph Selective Agar, and presence of pigment signatures associated with S. aureus
   (iv) K. pneumonia: Distinct color on CromUTI Agar, Strep Selective Agar, and Darkening of Bile Esculin Agar
   (v) P. mirabilis: Distinct color changes on Urea Agar
   (vi) A. baumannii: Distinct color changes on Acinetobacter Agar
   (vii) Enterococcus: Darketing of Bile Esculin Agar and distinct color changes on CromUTI Agar and Strep Selective Agar. No pigment production in GBS Medium (Carrot Broth)
   (viii) Group B Strep (S. agalactiae). Distinct pigment production in GBS Medium (Carrot Broth), color changes in Strep Selective Agar and CromUTI Agar, and no darketing of Bile Esculin Agar
   (ix) Candida organisms: Distinct color changes on CromCandida Agar, and darkening of Bile Esculin Agar
   (x) Mucor Organisms: Fibrous growth on CromCandida Agar and on Bile Esculin Agar, and Red Coloration on Bile Esculin Agar limited to the microorganism zone of growth.
34. The method according to any one of 24-33, wherein the method further comprises performing steps (i)-(iii) for a third fluid comprising the microorganism and the pharmaceutical drug at a concentration different than the pharmaceutical drug concentration in the first fluid.
35. The method according to any one of 24-34, wherein the microorganisms are bacteria.
36. The method according to any one of 24-35, wherein the power function has an order of −2, −3, or −4, wherein the order can be same or different between each of the optional repetitions.
37. The method according to any one of 24-36, wherein steps (A)-(C) are repeated 1 time, 2 times, or 3 times.
38. The method according to any one of 24-37, wherein the determining comprises comparing the rate of change in the two-dimensional plot of the biological fluid to a preset threshold.
39. The method according to any one of 24-38, wherein the detection component changes its optical absorbance in response a change in pH.
40. The method according to any one of 24-39, wherein the detection component changes its optical absorbance in response to an enzyme produced by a bacteria.
41. The method according to 40, wherein the enzyme is a urease enzyme, wherein the contacting step further comprises contacting the fluid with urea.
42. The method according to any one of 24-41, wherein the detection component comprises phenol red.
43. The method according to any one of claims 24-42, wherein the biological fluid is blood or urine.
44. A method according to treating a subject suspected to have an infection, comprising: performing or having performed the method of any one of 2-43.
45. The method of 45, further comprising administering an antibiotic to the subject.
46. A system for separating an absorption spectrum into a Rayleigh scattering contribution and an absorption contribution, comprising:

a light source;

a detector; and

a processor comprising memory operably coupled to the processor wherein the memory comprises instructions stored thereon, which when executed by the processor, cause the processor to:
irradiate a sample with the light source;
record an absorption spectrum with the detector; and
separate the absorption spectrum into a Rayleigh scattering contribution and an absorption contribution.
47. The system of 46, wherein the processor comprises memory with instructions for separating the absorption spectrum into a Rayleigh scattering contribution and an absorption contribution by:

(i) measuring an absorption spectrum;

(ii) generating a fit spectrum by fitting the absorption spectrum to a power function;

(iii) generating a difference spectrum by subtracting the fit spectrum from the absorption spectrum;

(iv) generating an adjusted spectrum by selecting;

points from the absorption spectrum for wavelengths wherein the difference spectrum is less than or equal to zero, and

points from the fit spectrum for wavelengths wherein the difference spectrum is greater than zero;

(v) repeating steps (ii)-(iv) zero or more times, wherein the most recent adjusted spectrum is used in place of the absorption spectrum if the steps are repeated, wherein the final adjusted spectrum is the Rayleigh scattering contribution; and

(vi) generating the absorption contribution by subtracting the Rayleigh scattering contribution from the absorption spectrum.
48. The system of any one of 46-47, wherein instructions are configured for irradiation, recordation, and separation for a plurality of samples.
49. A non-transitory computer readable storage medium, comprising instructions stored thereon for separating an absorption spectrum into a Rayleigh scattering contribution and an absorption contribution, the instructions comprising:

(i) an algorithm for measuring an absorption spectrum;

(ii) an algorithm for generating a fit spectrum by fitting the absorption spectrum to a power function;

(iii) an algorithm for generating a difference spectrum by subtracting the fit spectrum from the absorption spectrum;

(iv) an algorithm for generating an adjusted spectrum by selecting: points from the absorption spectrum for wavelengths wherein the difference spectrum is less than or equal to zero;

points from the fit spectrum for wavelengths wherein the difference spectrum is greater than zero;

(v) an algorithm for repeating steps (ii)-(iv) zero or more times, wherein the most recent adjusted spectrum is used in place of the absorption spectrum if the steps are repeated, wherein the final adjusted spectrum is the Rayleigh scattering contribution;

(vi) an algorithm for generating the absorption contribution by subtracting the Rayleigh scattering contribution from the absorption spectrum.
50. A system comprising:

an n-well plate reader (where n is 6, 12, 48, 96 or 384), that are prefilled with various reagents and antibiotics, and to which a fixed amount of the test sample is added.

a tunable microplate reader that accepts the n-well plate and which acquires a UV-Vis absorption spectrum from all n wells upon the instruction to do so being provided by a microcontroller;

a microcontroller or a computer with software with a suitable connection to the tunable microplate reader that can instruct the microplate reader to acquire a UV-Vis absorption spectrum at a preset spectral resolution, and in a preset spectral range, and which can also acquire the data collected by the tunable microplate reader; and

a microcontroller that can implement the methods according to any one of claims 2-45.
51. A system for implementing the methods according to any one of 2-45, the system comprising one or more of:

n-well plates;

petri dishes;

petri dish biplates; and

petri dish quadplates.
52. The system according to 51, wherein one or more of the n-well plates, petri dishes, petri dish biplates and petri dish quadplates are prefilled with the suitable reagents.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); and the like.

### Example 1: Detecting Changes in Phenol Red to Characterize Bacteria Presence

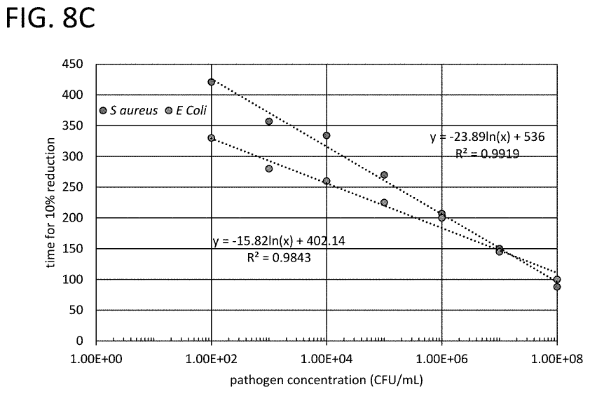

The presence of any bacteria in a test sample was detected. With rare exceptions, the metabolic activity of bacteria produces pH lowering metabolites. When combined with a pH indicator molecule (like phenol red), the pH lowering metabolites decreases the height of the phenol red absorbance peak at 560 nm. Alternatively, the phenol red peak at 440 nm can also be considered. Alternatively, other pH indicator molecules can also be used. The measured height of the phenol red absorbance peak is compromised by several factors, such as the presence of microbubbles in the liquid sample, the migration of these microbubbles in the optical path, the presence of various other protein aggregates, and the aggregation of these protein aggregates in the optical path. These artifacts can compromise the measured phenol red peak height, and thus impede the detection of bacteria presence. For the most part, such artifacts affect the Rayleigh scattering contribution. Thus, the present disclosure combines methods that accurately recognize the phenol red peak height, as described herein. Embodiments described in FIG. 7, and uses the disclosures herein to accurately detect the phenol red peak heights for 6 samples, including 3 “Infected” samples with 1000 CFU/mL of S. aureus and 100 CFU/mL “E. coli”, 3 “control” samples with 100 CFU/mL E. coli. With these peak heights, the system computes the ratio of the absorbance peak for the 3 infected and the 3 control samples. This ratio decreases over time, and a significant decrease (which we define as when the linear fit to the datapoints has a negative slope that is greater than the 95CI around the slope) denotes the presence of some bacteria in the “Infected” sample at a concentration that is significantly greater than the concentration of bacteria in the control sample. In this example, bacteria presence can be discerned at 63 minutes. A second approach is to consider the decrease in the phenol red peaks for control and infected samples, the difference in these two as a signal, and the rms standard deviation as a noise. This SNR (signal divided by noise) builds up exponentially over time, starting from a value of 0, as illustrated in FIG. 7. When the SNR metric exceeds a preset threshold of 1, this can be taken as an additional confirmation of bacteria presence. In this example, bacteria presence is confirmed at 240 minutes. Thus, the innovations described here allows bacteria detection in 63 minutes, and confirmation of bacteria presence in 4 hours; compared to the overnight growth normally involved for detecting bacteria presence with standard phenol red broth detectors.

### Example 2: Quantifying the Bacteria Concentration

Quantification of bacteria concentration in a test sample was performed. As described herein, the metabolic activity of bacteria produces pH lowering metabolites that reduces the height of the phenol red peak at 560 nm, and increases the height of the phenol red peak at 440 nm. Also, as described herein, other suitable pH indicator molecules can be used for this detection.

Thus, the present disclosure combines methods that accurately recognize the phenol red peak height, as described herein, with additional innovations to accurately quantify the bacteria concentration. FIGS. 8A-C summarizes the phenol red 560 nm peak output as a function of time, for samples comprising 50 uL of a phenol red solution, 125 uL of 1× TSB (formulated in water), and 50 uL of a test sample (formulated in PBS buffer) with a varying concentration of bacteria (E. coli 25933 in this example). The figure illustrates the following changes: (1) As show in FIGS. 8A, in the uninfected control samples, there is a slight reduction in the 560 nm peak height, reflective of changes in temperature, or evaporation. Thus, all changes from a test sample are considered in relation to the changes in the control sample (2) As show in FIG. 8B, once the 560 nm peak from a test sample is normalized with the corresponding value from the control sample, then the changes are due to acid production by the bacteria. This acid production result in a sigmodial decrease in the phenol red peak height. (3) As show in FIG. 8C, the time required for a 10% reduction in the phenol red peak height shows a correlation with the logarithm of the pathogen concentration in the test sample. There is a range of values, accounting for the different in growth rates of different bacteria. Thus, if the ID of the bacteria is not known, then for a given time required for 10% reduction, the bacteria concentration can be estimated to within a factor of 10. For instance, if the time required for 10% reduction is 300 minutes, then the bacteria concentration can range from 500 to 20,000 CFU/mL. If the ID of the bacteria is known, then the corresponding pathogen concentration can be inferred more accurately. For instance, if the time required for 10% reduction is 300 minutes and the bacteria is known to be E. coli, then the bacteria concentration can range from 500 to 2,000 CFU/mL. To accurately estimate we also need to ensure that the control sample is truly free of any bacteria because it will be used to normalize away the changes due to thermal drift. This can be ensured by subjecting the test samples to a microwave induced heating step, and sealing the individual wells with strong transparent packaging tape to eliminate the possibility of well-to-well cross contamination. For our 96 wells, we have determined that a microwave heating step of 30 second duration is sufficient to sterilize the contents of each well. Accordingly, the process steps are as follows: (1) lead each well with 125 uL of 1× TSB, and 50 uL of the phenol red solution. (2) Seal with packaging tape (3) Microwave 96 well plate at 1000 W for 30 seconds. (4) Let cool to room temperature for about 5 minutes. (5) Remove packaging tape and add test sample (6) Reseal with packaging tape, and place 96 well plate in a plate reader with a sample chamber set to 37° C. (7) Collect spectra and process into color spectrum as described herein (8) Compute the 560 nm peak heights from the test sample, normalize with the corresponding value in the control sample and compute the net reduction. (9) Compute the time at which this quantity is reduced by 10%. (10) Read the pathogen concentration from the logarithmic dependence described in FIG. 7 bottom panel.

Accurate detection of the phenol red peak heights for 6 samples, including 3 “Infected” samples with 1000 CFU/mL of S. aureus and 100 CFU/mL “E. coli”, 3 “control” samples with 100 CFU/mL E. coli. With these peak heights, the system computes the ratio of the absorbance peak for the 3 infected and the 3 control samples. This ratio decreases over time, and a significant decrease (which we define as when the linear fit to the datapoints has a negative slope that is greater than the 95CI around the slope) denotes the presence of some bacteria in the “Infected” sample at a concentration that is significantly greater than the concentration of bacteria in the control sample. In this example, bacteria presence can be discerned at 63 minutes. A second approach is to consider the decrease in the phenol red peaks for control and infected samples, the difference in these two as a signal, and the rms standard deviation as a noise. This SNR (signal divided by noise) builds up exponentially over time, starting from a value of 0, as illustrated in FIG. 7. When the SNR metric exceeds a preset threshold of 1, this can be taken as an additional confirmation of bacteria presence. In this example, bacteria presence is confirmed at 240 minutes. Thus, the innovations described here allows bacteria detection in 63 minutes, and confirmation of bacteria presence in 4 hours; compared to the overnight growth normally required for detecting bacteria presence with standard phenol red broth detectors.

### Example 3: Detecting Changes in Phenol Red with a Urea Broth Base to Characterize Urease Producing Bacteria Presence

The presence of any urease producing bacteria in a test sample was detected. Normally, the metabolic activity of bacteria produces pH lowering metabolites. One exception to this is for bacteria that produce the Urease enzyme, and when the broth medium contains Urease as the primary source of carbon and nitrogen. In this case, the Urea is hydrolyzed with ammonia as a byproduct, thereby raising the pH of the solution/test sample. If a pH indicator molecule (like phenol red) is present in the solution, the pH increase results in an increase in the absorption peak at 560 nm. With a long enough incubation time (about 18-24 hours), the increase in the 560 nm absorbance is significant enough to be apparent to the naked eye.

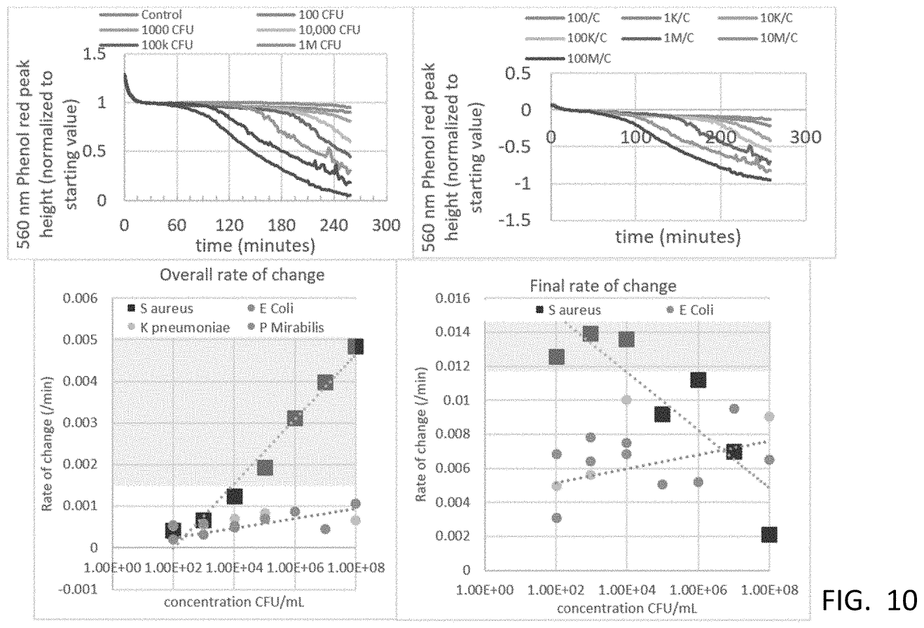

This reading was conducted in about 2-3 hours of incubation, and is illustrated in FIG. 9. The chart on the top left illustrates the difficulty in reading the color change at short periods (at 4 hours in this example). The color spectra is dominated by the Rayleigh/Phenol red contributions, and the changes are relatively small (even at 4 hours). To discern the color spectrum, we use the methods described herein to separate the “Change” into a change in the Rayleigh contribution (that scales as the inverse 2nd power of wavelength), and a change in the Color spectrum. The change in the color spectrum can be integrated over 500-600 nm (i.e., covering the expected 560 nm absorbance peak), and this grows exponentially over time as illustrated in the figure. Thus, when an exponential fit to this metric has a damping coefficient greater than 0, and is also greater than the confidence interval around this estimate, then this signifies the presence of Urease producing bacteria. In this example, Urease production is detected at 140 minutes.

The value of the disclosure described herein can be understood by comparing the time required to detect Urease production with various factors that are detuned. If the methods described herein are modified to include a general polynomial fit for the Rayleigh contribution, then it takes over 5 hours to detect Urease production. If the methods described in described in herein are not used, and the change in 560 nm absorbance is detected with 2 point comparisons (between 560 nm and 630 nm, for instance), then it takes 4 hours to detect Urease production.

Methods also include determining the presence or absence of a microorganism in a sample as well as for determining the signal-to-noise ratio and correcting for thermal drift of a monochromatic light source. Systems for practicing the subject methods are also provided.

### Example 4: Staphylococcus Specific Medium with Phenol Red

The presence of any urease producing bacteria in a test sample was detected. The methods described herein rely on acid production by metabolic activity of bacteria. Acid production is allowed by a media, which was trypticase soy broth herein. These methods can be modified to include a specific media. For instance, the mannitol-salt medium comprises a high salt content that is tolerated by Staphylococcus organisms, and mannitol that is fermented by Staphylococcus and E. coli. Accordingly, the mannitol-salt medium with phenol red is used to distinguish the presence of Staphylococcus. For instance, Hardy Diagnostics sells a Mannitol Salt agar (https://catalog.hardydiagnostics.com/cp_prod/Content/hugo/MannitolSaltAgar.htm), plates on which S. aureus grows into luxuriant yellow colonies, S. epidermidis grows into red colonies, and other bacteria (like Proteus mirabilis and Escherichia coli) do not grow. However, this discrimination normally takes over 24 hours of growth. The methods described herein allow for this discrimination in less than 4 hours.

FIG. 10 illustrates these methods. We use the mannitol-salt-phenol red medium available from HiMedia (https://www.himediastore.com/mannitol-salt-broth-6074). We create samples by mixing up the media as per the manufacturer's directions, and adding 150 uL of the media to individual wells in a 96 well plate. To those wells, we add 50 uL of a test sample that are all formulated in 1× PBS buffer and that is either a control sample, or with varying amounts of bacteria. We monitor the height of the phenol red peak using a 96 well plate reader and the methods herein to separate out the changes in phenol red output. With these methods, the phenol red output can be seen to start decreasing significantly after about 1 hour for 108 CFU/mL S. aureus, and about 4 hours for 102 CFU/mL S. aureus. Thus, the specific presence of S. aureus can be gauged by either the overall rate of change of the phenol red 560 nm peak, or the final rate of change of the phenol red 560 nm peak being in the diagnostic band.

### Example 5: Staphylococcus aureus Specific Detection

Aspects of the present disclosure include methods and systems for detecting the presence of Staphylococcus aureus in test samples. S. aureus is a unique pathogen in that it makes a series of triterpenoid carotenoids that are said to be related to it's virulence. (Reference: “Pigments of Staphylococcus aureus, a Series of Triterpenoid Carotenoids” Marshall & Wilmoth 1981 https://jb.asm.org/content/jb/147/3/900.full.pdf) The absorption spectrum of these triterpenoids comprises a triplet absorption spectrum (see FIG. 3 in Marshall & Wilmoth 1981) with one of the peaks in the triplets centered at 488 nm.

Aspects of the present disclosure includes the observation that all S. aureus strains (including wild type strains found in clinical samples) will produce these triterpenoid carotenoids, when incubated in the “proper” media, and with “sufficient” oxygen in the media. “Proper” media includes at least two examples: The CromUTI Agar available from HiMedia Laboratories (https://www.himedialabs.com/intl/en/products/Clinical-Microbiology/Diagnostic-Media-for-Bacteria-Klebsiella/HiCrome%E2%84%A2-UTI-Agar-SM1353) and the Streptococcus Selective Agar, also available from HiMedia Laboratories (https://himedialabs.com/TD/M1840.pdf; used without the Selective Agents recommended in the formula). “Sufficient” oxygen refers to the amount of dissolved oxygen contained in the Agar media. Such Agar based formulations are sterilized by autoclaving (>121° C., >15 psi>15 min), wherein the amount of dissolved oxygen is depleted. The Agar media is poured (or pipetted) by first cooling to 50° C. “Sufficient” oxygen is enabled when the Agar media is held at 50° C. for 1 hour, and the plates are used after a further 48 hour hold at room temperature.

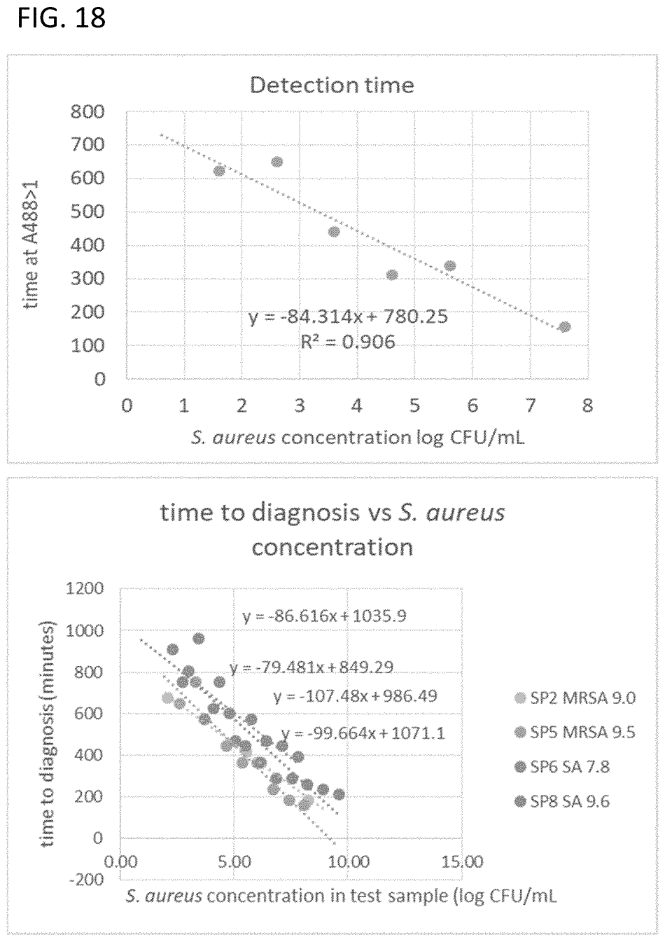

Under these conditions of “proper” media and “proper” oxygen, wild type and ATCC strains of S. aureus produce the triterpenoid toxin that can be used to characterize the presence of S. aureus. FIG. 17 depicts the UV-Vis absorption spectrum collected from a Crom-UTI agar (0.15 mL of the media poured into a well in a 96 well plate) which a test sample (0.05 mL) containing the ATCC strain of S. aureus (at 10⁴ CFU//mL) has been added. The measured spectrum (shown on the left in FIG. 17) comprises a “color” spectrum and the “Rayleigh” contribution. The measured spectrum is not that useful as is, but when the methods described in

are used to separate out the Rayleigh scattering component, then the remainder (ie, the “color” spectrum; shown on the right in FIG. 17) resembles absorption spectrum of the triterpenoid described by Marshall and Wilmoth 1981. The color spectrum can now be recognized as due to S. aureus by using simple algorithms to threshold the presence of one, or more of the 3 peaks described in FIG. 17.

Aspects of the present disclosure further include methods to recognize the “color” absorption spectrum depicted in FIG. 17 as being due to S. aureus. With respect to the triplet described in FIG. 17, the presence of any one of the peaks in the triplet can be associated with S. aureus presence. For example, considering the peak centered at 488 nm. A metric S computed as 2×A₄₈₈/[A₄₇₅+A₅₀₅] would indicate S. aureus presence when S>1. In the metric S, A₄₈₈, A₄₇₅ and A₅₀₅ refer to the absorbance in the color spectrum (ie, after the removal of the Rayleigh contribution using the methods described in [0041]. Thus, the diagnosis of S. aureus presence can be made with a set of subsystems that collects the UV-Vis absorption spectrum from the CromUTI media and test sample, computes the “color” spectrum, and the metric S. If this metric S exceeds 1, then this indicates S. aureus presence. When measurements are initiated, the metric S is generally less than 1. As S. aureus produces the triterpenoid pigment, the metric S increases above 1.

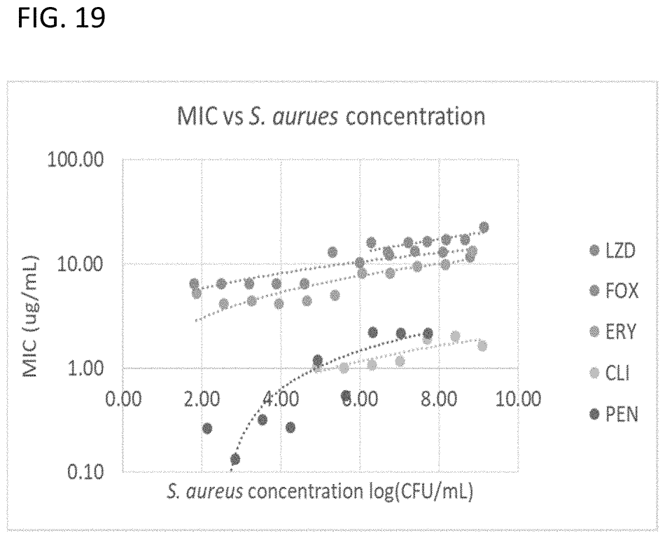

Aspects of the present disclosure further include methods to characterize the S. aureus concentration. As depicted in FIG. 18, the time at which the metric S exceeds 1 scales with S. aureus concentration. We find a similar scaling behavior for a number of wild type S. aureus strains. Thus from the time at which the metric S exceeds 1, the S. aureus concentration in the test sample added to the CromUTI agar can be estimated using the relationship depicted in FIG. 18.

### Example 6: Characterizing Response to Candidate Antibiotics

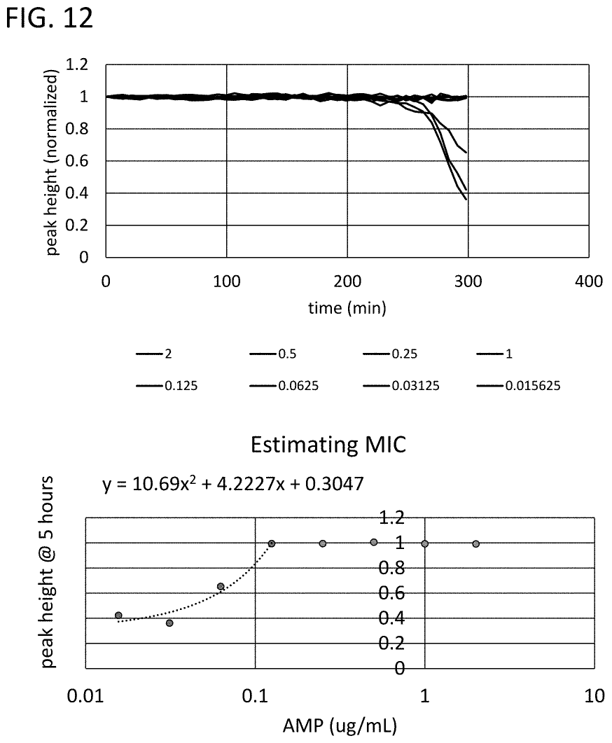

Antimicrobial susceptibility response was characterized. The metabolic activity of the causative pathogen can be suppressed by an effective antimicrobial. Thus, when a candidate antibiotic, present at a test concentration, is combined with the teachings described herein, then we can deduce the effect of that antibiotic on the microorganism. If we combine a series of antibiotic concentrations, then we can deduce the minimum inhibitory concentration. This is illustrated in FIG. 11 for the case of E. coli 25922 (a quality control strain of E. coli) against Gentamicin GM. In this example, the test solutions had a Gentamicin concentration that started at 2 μg/mL, and decreased in intervals of 2×. We find that the 560 nm phenol red absorbance peak is substantially suppressed for all test solutions, other than the one at 2 μg/mL. A linear fit applied to the phenol red peak height results in an extrapolated value of 2.03 μg/mL as the GM concentration at which the 560 nm peak height does not decrease from the starting value of 1.

Other specific implementations of the scheme above can also be realized by those skilled in the arts. For instance, the media can be replaced by Cation Adjusted Mueller Hinton Broth CAMHB, which makes the process consistent with the CLSI M100 specified process. Further, instead of reading bacteria growth by changes in the phenol red peak, the Rayleigh scattering can be directly read as a signal for bacteria growth. If the starting concentration of bacteria is low enough (i.e., below the saturation threshold of about 10⁷ CFU/mL), then the Rayleigh scattering absorbance will increase with bacteria concentration). Use of lower concentrations of bacteria in the test solution (compared to the 0.5 McFarland, or 2×10⁸ CFU/mL specified in the CLSI M100 specification) will require a correction (described in Example 7) to estimate a rapid CLSI equivalent MIC. Finally, the threshold for MIC can be set at some arbitrary reduction in growth that is designed to ensure maximum concordance with CLSI Methods (after the correction described in Example 7 below).

### Example 7: Estimating & Correcting MIC for Pathogen Concentration

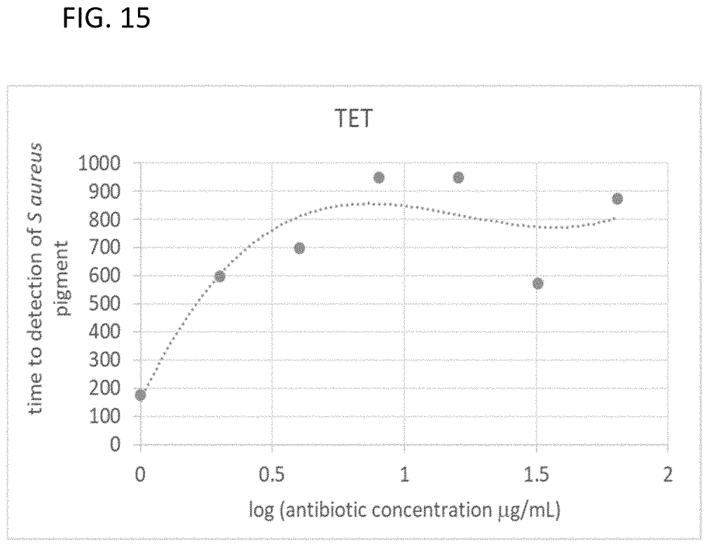

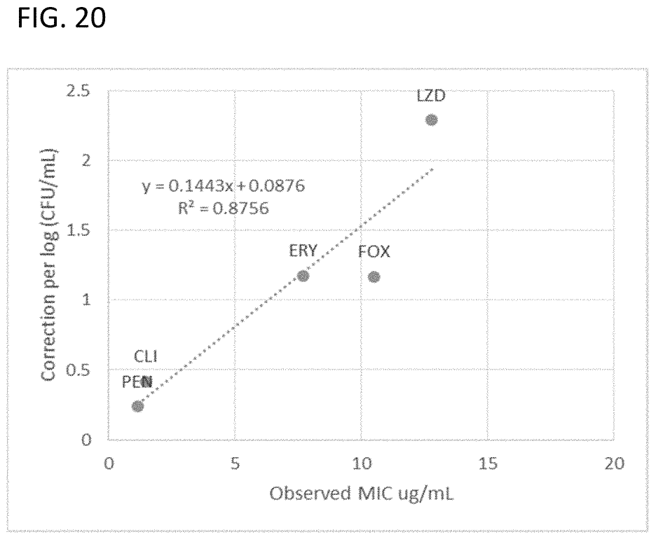

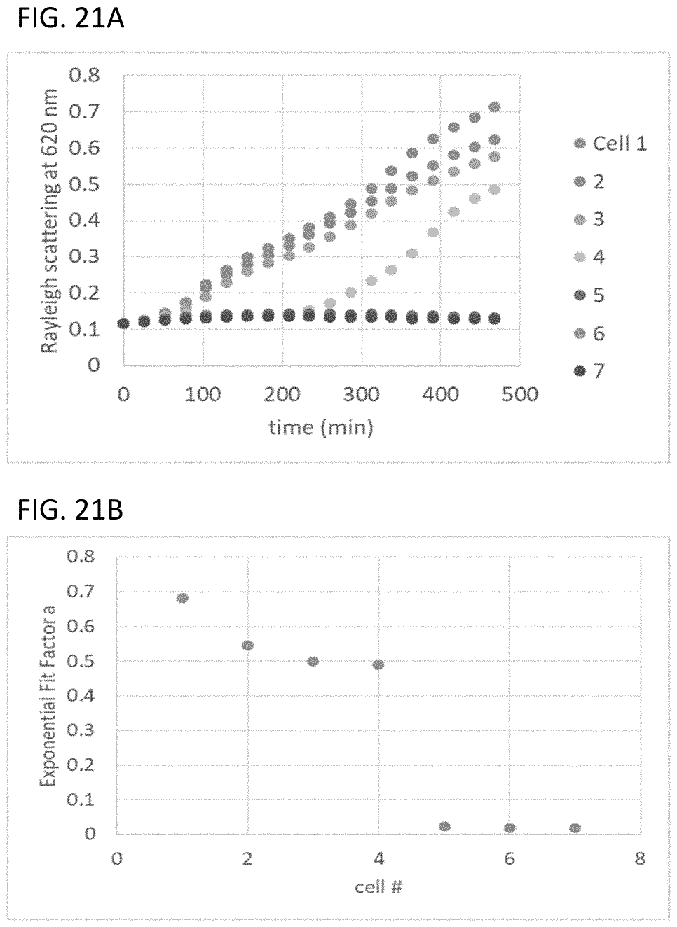

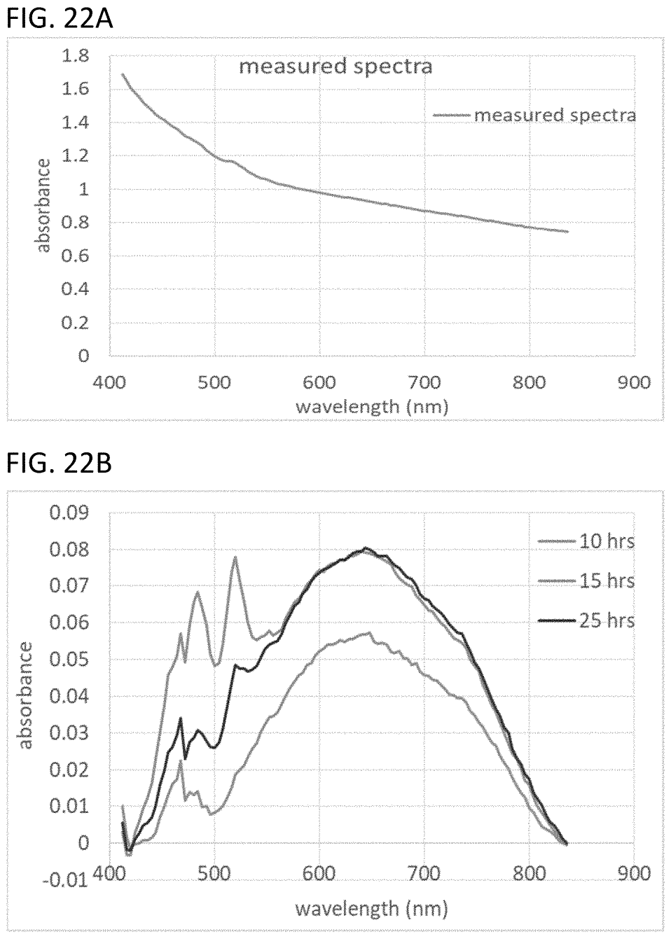

As we observe experimentally, the antimicrobial susceptibility metric MIC is a function of pathogen concentration, as depicted in FIG. 19. Thus, estimates for MIC obtained from a test sample that is at a pathogen concentration lower than the concentration of 2×10⁸ CFU/mL (or 0.5 McFarland) specified in the CLSI M100 standard will need to be corrected for this variation to maximize concordance between a rapid test MIC and the CLSI standard. Empirically, we find that the slope of the traces depicted in FIG. 19 scales with the absolute magnitude of the estimated MIC, as depicted in FIG. 20. Thus, an algorithm to correct the MIC for pathogen concentration is to use the empirical observed scaling relationship depicted in FIG. 20, along with the pathogen concentration estimated from the methods described in FIG. 18. As illustrated with the example for FIG. 21, the MIC that is estimated at the test pathogen concentration is 0.192 μg/mL. Separately, the time to detection is measured as 161 minutes, and this returns an S. aureus concentration of 7.5×10⁶ CFU/mL. Using this concentration, and the equation of FIG. 14, we estimate that the CLSI M100 MIC at 0.5 McFarland will be 0.867 μg/mL.

### Example 8: Using MIC, Bacteria Concentration and Bacteria ID to Determine if Pathogen is Resistant or Susceptible to a Candidate Antibiotic

Once the antimicrobial susceptibility metric MIC and bacteria concentration are determined, and the MIC is corrected for the variation in MIC with bacteria concentration such that an MIC at a McFarland 0.5 is estimated, the MIC can be compared with the breakpoints listed in the CLSI M100 manual to determine if the bacteria is resistant or susceptible to the candidate antibiotic.

We illustrate this process with one example of a wild type strain of S. aureus tested against Vancomycin in Cation Adjusted Mueller Hinton Broth (CAMHB Himedia labs M1657). With CAMHB, the viability of the bacteria is estimated by plotting the Rayleigh scattering factor versus time, and fitting this to an exponential growth function a/[1+exp(−(t−b)/c)], as illustrated in FIG. 21. The growth factor a is then plotted against antibiotic concentration, and from this profile, the concentration at which a approaches zero is estimated as the nominal MIC at the pathogen concentration. Separately, the pathogen concentration is estimated and the MIC is corrected for the pathogen concentration. This results in an CLSI M100 MIC estimate of 0.867 μg/mL. The CLSI M100 breakpoints for Staphylococcus against Vancomycin are >16 μg/mL for resistant and <2 μg/mL for susceptible. Accordingly, this strain of S. aureus is reported as being susceptible to vancomycin,

Variations of the above approach can also be adopted with the teachings described here. For instance, instead of using Cation Adjusted Mueller Hinton Broth, we can also use CromUTI agar (or Strep Selective Agar). The CromUTI Agar enables S. aureus pigment production, and the timeline of production of pigment can be used as an indicator of antibiotic effectiveness. This approach has the advantage of focusing the antibiotic response and bacteria ID on the same well, thereby enabling testing on polymicrobial samples. However, CromUTI Agar is also known to dry out over time, and it becomes a less effective medium as it dries out, thus higher noise metrics are expected.

### Example 9: Detecting the Presence of Streptococcus agalactiae (Group B Strep)

Group B Streptococcus (S. agalactiae) is known to produce a carrot colored pigment when incubated in a “carrot broth” (available from several vendors, we used GBS Medium from HiMediaLabs https://himedialabs.com/TD/M1073.pdf). The carrot-red pigment is said to be produced by about 97% of all GBS strains and is associated with maxima in the UV-Vis absorption spectrum at 435, 566, 485, and 525 nm (reference: https://pubmed.ncbi.nlm.nih.gov/353069/). The procedure described for characterizing pigment production is fairly elaborate, and involves centrifuging the GBS cells and pigments, and washing the centrifuge pellet in various solvents to extract the pigment into a solvent that is suitable for UV-Vis measurements. To our knowledge, no teaching describe methods whereby the GBS pigment can be detected directly from the growing solution. Absent spectroscopic identification, current laboratory practice includes a visual observation of the carrot color after incubation in the “carrot broth” medium for 24 hours.

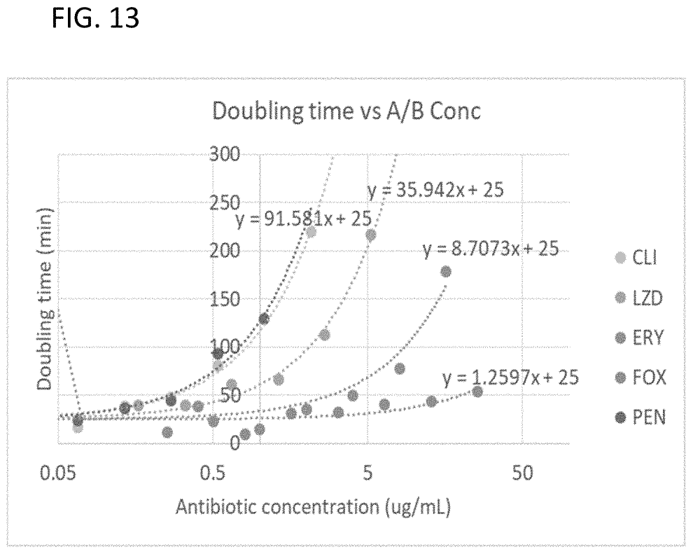

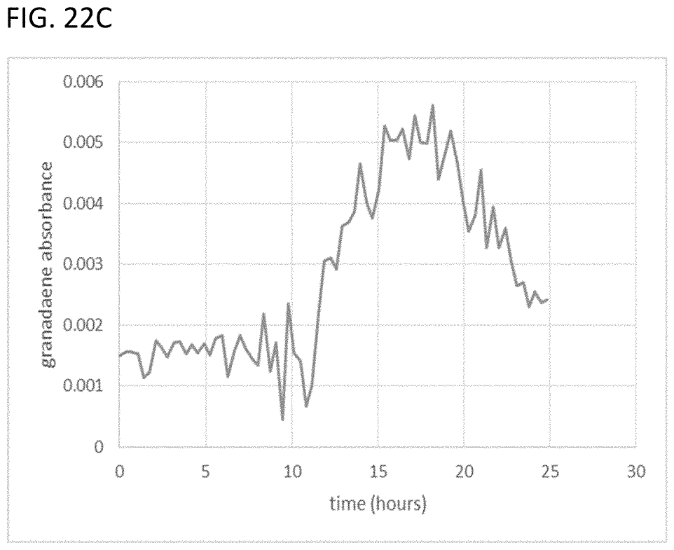

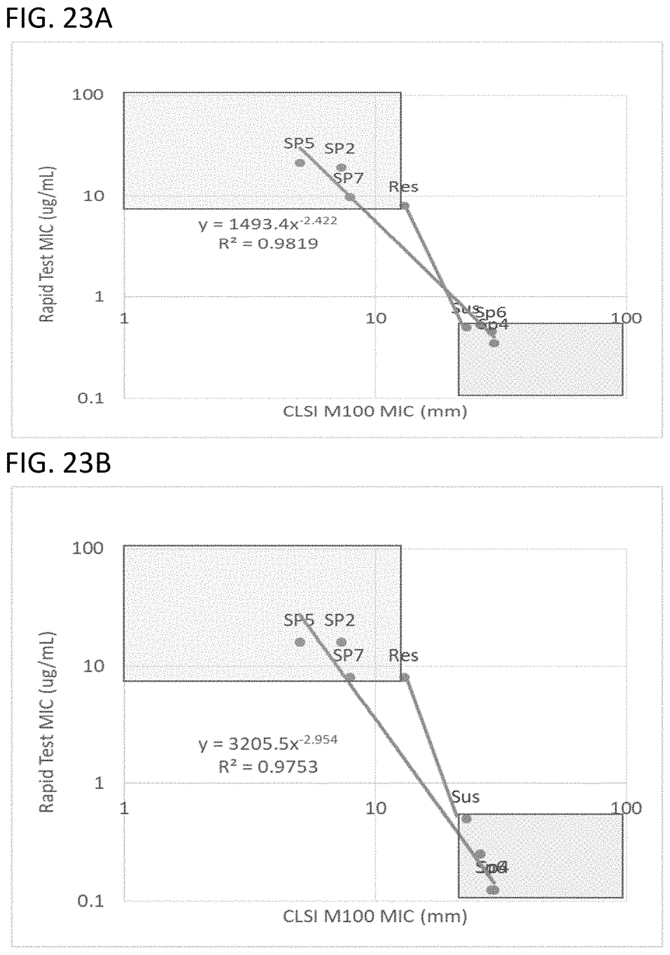

Aspects of the present disclosure include methods to characterize GBS pigment production in a suspension that includes the test bacteria suspension and the GBS medium without having to resort to centrifuging and extraction in a solvent. The difficulty in such measurements results from the UV-Vis spectrum being dominated by the Rayleigh contribution, as is illustrated in the example for S. aureus in FIG. 12, and for GBS in FIG. 22. Using the same methods as those described for the S. aureus pigment in FIG. 12, the pigments produced by GBS can be detected directly in a test sample that includes the GBS medium and the bacteria. This is illustrated in FIG. 22, for the medium and test bacteria incubated at 37 C in a 96 well plate.

### Example 10: Implementing Pathogen ID and Antimicrobial Susceptibility on a 96 Well Plate and Tunable Microplate Reader

Aspects of the present disclosure include methods and systems for characterizing the ID of a test bacteria (including characterizing the presence of more than one bacteria in a polymicrobial sample) and characterizing the response of the test bacteria to a set of candidate antibiotics. The subsystems for this include a 96 well plate filled with several reagents, and a tunable 96 well plate reader for measuring the absorbance spectrum from those 96 well plates after a test suspension of bacteria has been added to it.

The example described here includes 12 reagents that are listed here. (1) ChromUTI Agar (Catalog M1353 from Himedia Labs) (2) ChromCandida Agar (Catalog M1297 from Himedia Labs) (3) Staph Selective Agar (Catalog M1931 from Himedia Labs) (4) Aureus Tellurite (Catalog M1468 from Himedia Labs) (5) MM Agar (M1393 from Himedia Labs) (6) Strep Selective Agar (M1840 from Himedia Labs) (7) GBS Medium (M1073 from Himedia Labs) (8) Acinetobacter Agar (M1839 from HiMediaLabs) (9) HiColiform Agar (M1453 from Himedia Labs, formulated as per manufacturers instruction, and to which standard Agar is added to formulate the media into an agar) (10) MacConkey Sorbitol (11) Urea Agar (12) Bile Esculin Agar (M493 from Himedia labs)

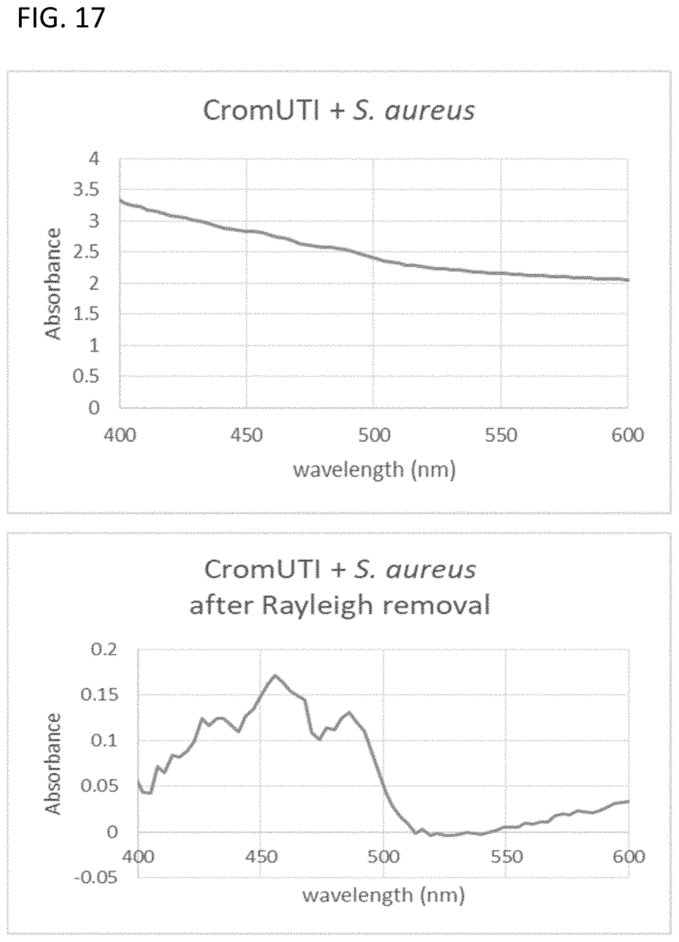

Other combinations of reagents can be used to achieve essentially the same purpose. The ChromUTl reagent is useful for characterizing the dominant organism in the test sample, and provides a color change that differs for Staphylococcus vs E. coli vs Group B Strep/Enterococcus. FIG. 16 depicts the color spectra from 5 different bacteria. The spectra from some bacteria are more uniquely identifiable than from others. For instance, the spectra from Enterococcus (example E. faecalis depicted in FIG. 16) and S. agalactiae are similar to each other, but very different from Staphylococcus and E. coli. On the other hand, the spectra from S. aureus includes a distinct pigmentation, as already described in Example 7. And the color from E. coli includes a maxima at about 550 nm that is fairly unique. Other example of color specific media in the set of 12 reagents we use are the CromCandida Agar (which provides for two different colors with various Candida organisms), MM Agar (which provides for 4 different types of color changes),

One of the reagents (the ChromCandida Agar) is designed to provide a color change for Candida type organisms. Color changes on this, combined with a black coloration on the Bile Esculin Azide Agar signifies Candida organism. The Staph Selective Agar and the Aureus Telluride Agar are designed to signify the presence of Staphylococcus (a common bacterial pathogen found in blood). The Strep Selective Agar, as supplied by the vendor, is designed for use with certain selective agents that makes the Agar selective to Group B Streptococcus (GBS S. agalactiae). We use it without the selective agent˜doing so enables a color change for both GBS and Enterococcus (visually there is a blue color, and on the plate reader, the color spectrum resembles the color spectrum depicted in FIG. 16 for ChromUTl agar. A positive on the Strep Selective Agar, combined with a negative on the Bile Esculin Azide agar will generally imply the presence of S. agalactiae. (however, a positive on the Bile Esculin Azide Agar and a positive on the Bile Esculin Azide agar does not provide sufficient information on the presence/absence of S. agalactiae). The GBS Medium (commonly referred to as the carrot broth) provides information on the presence of S. agalactiae via the production of the GBS pigment (described in Example 9). As per previous clinical studies, this medium detects about 97% of all clinical strains of GBS. With continuous monitoring of the GBS pigment, we believe that this method will detect pigment production from 100% of all clinical strains of GBS. The HiColiform broth is designed to provide a response specific to E. coli˜the color changes are due to an enzyme produced by E. coli. The Mac-Sorb Agar provides a means to distinguish pathogenic strains of E. coli (which cannot ferment sorbitol) from the non pathogenic strains (which do ferment sorbitol). Fermentation is indicated by color changes in the pH indicator contained in the medium. The Urea Agar indicates the presence of Urea fermenting bacteria (P. mirabilis is an example). He presence of Urease producing bacteria is indicated by an increase in pH that results in distinct color changes in the pH indicator.

Aspects of the present invention include media with varying concentrations of a candidate antibiotic. In one embodiment, we use the following 12 antibiotics: Vancomycin (VAN) Tetracycline (TET) Penicillin (PEN) Clindamycin (CC) Erythromycin (ERY), Cefoxitin (FOX), Linezolid (LZD), Gentamicin (GM), Ciprofloxacin (CIP), Tobramycin (NN) Imipenem (IMP) and Cefazolin CFZ. Other combinations of antibiotics can also be selected, while using the teaching deployed here. These antibiotics are selected so as to provide some coverage against common pathogenic microorganisms S. aureus, S. epidermidis, S. agalactiae, E. faecalis, and E. coli. For each antibiotic, we use 7 concentrations that range from some multiple (generally about 1 to 2×) of the CLSI M100 resistant breakpoint (for that antibiotic and the targeted pathogen), and decrease in factors of 2 down to a concentration that is below the CLSI M100 susceptible breakpoint (for that antibiotic/pathogen combination). For instance, for VAN, the CLSI M100 breakpoints for staphylococcus are 16 and 2 μg/mL. Accordingly, we use antibiotic concentrations of 32, 16, 8, 4, 2, 1 and 0.5 μg/mL.

Each antibiotic concentration is prepared in a unique well in a 96 well plate. In the configuration above, we use 12 antibiotics at 7 concentrations each; thus using 84 wells for AST measurements and 12 wells for bacteria ID as described in [00104]. This configuration is designed to develop the MIC values in a manner analogous to the CLSI M100 methods. Other arrangement can also be used, using the teachings described here

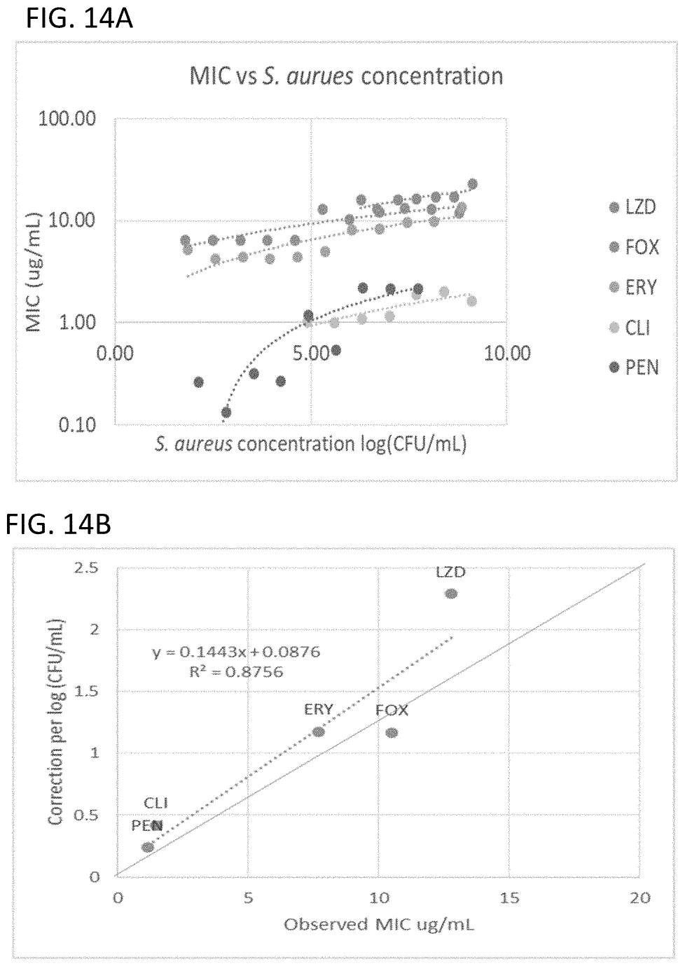

Estimating the CLSI M100 MIC via a rapid test is difficult due to two factors: (a) First, the pathogen doubling time increases exponentially as the antibiotic concentration increases (FIG. 13). At some concentration, the doubling time is expected to diverge. The CLSI M100 MIC refers to a concentration at which no growth is observed after 16-20 hours of incubation (24 hours for vancomycin) for incubation at 35±2° C., with a starting concentration of 0.5 McFarland (2×10⁸ CFU/mL). Thus, the CLSI M100 MIC refers to some concentration below the concentration at which the doubling time diverges in FIG. 13. Presumably, if the samples are incubated for 48 hours (instead of 16-20 hours), then growth could be observed at some antibiotic concentrations where no growth is observed at 20 hours. The exact position along the curve will depend on the pathogen concentration. Accordingly, the estimated MIC itself becomes a function of pathogen concentration when using methods analogous to the CLSI M100 methods but without using the 0.5 McFarland pathogen concentration. This is illustrated in FIG. 14. Thus, any estimate of MIC that is developed at a non-standard (ie other than 0.5 McFarland) pathogen concentration must be corrected for this effect. The variation in MIC with pathogen concentration is described with a linear fit (see FIG. 14 top), with the slope of the linear fit itself a linear function of estimated MIC (see FIG. 14 bottom). So the correction of MIC for pathogen concentration requires that the pathogen concentration be estimated independently (for example, using the methods described in Example 7) and then using the correction of FIG. 14, which is also described in Example 7

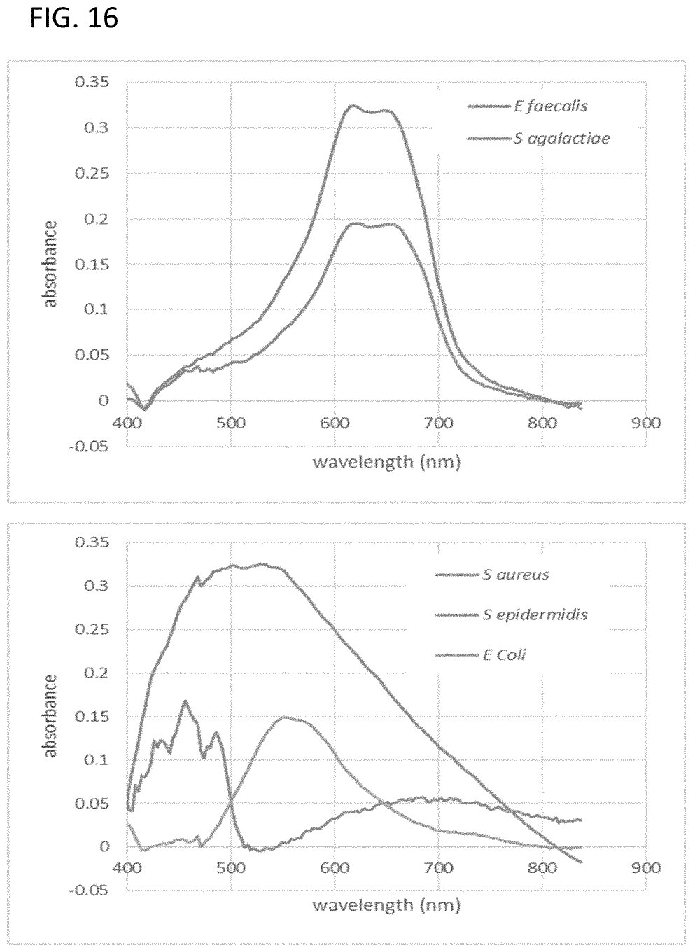

The second factor that complicates a rapid test for MIC is the presence of multiple pathogens in a polymicrobial sample. For example, a polymicrobial sample that is dominated by Enterococcus but in which the S. aureus is the pathogen of concern, the MIC estimated by examining the growth of Rayleigh scattering can be erroneous because of an incorrect correction for pathogen concentration, and because the Rayleigh scattering is dominated by Enterococcus growth. This issue can be mitigated by one of two ways: (a) by flagging all polymicrobial samples & (b) by focusing the response on a signal associated with the pathogen of interest. For example, using the methods described in Example 5, we detect the S. aureus specific pigments, and characterize the time required for the detection of these pigments as a function of antibiotic concentration. This is illustrated in FIG. 15 for a clinical sample containing fig, and the antibiotic tetracycline. From this variation, the MIC is estimated as the minimum antibiotic concentration required to increase the time to detection metric by 2× compared to the time to detection metric observed for the media without any antibiotic (which was 150 minutes in this example). And so, the MIC in this example is log (concentration)=0.3, or a concentration of 2 μg/mL. The concentration of S. aureus estimated from the detection time of 150 minutes (and the methods described in Example 8) is 8.8×10⁶ CFU/mL. Using this concentration, and the methods described in Example 7, the MIC at 0.5 McFarland is estimated as 2.51 μg/mL. This clinical sample also contained Enterococcus (which was evident after isolating individual colonies on a blood Agar plate), but the method described here focuses the MIC metric on the response of S. aureus.

### Example 11: Screening for the Presence of Mucorales with Visual Observations

The teachings described here can also be implemented for a diagnostic test using visual observations only. Some of the color changes associated with some reagents can be exploited in non-traditional ways. For instance, in the Bile Esclin agar, the Dark Red coloration is normally associated with hydrolysis of the glycoside esculin in the medium. When an organism hydrolyzes the glycoside esculin to form esculetin and dextrose, the esculetin reacts with the ferric citrate to produce a dark brown or black phenolic iron complex. Esculetin production is associated with the whole plate (if the media is poured on the plate) turning dark red because the esculetin diffuses away from the bacteria cells where it is produced. By contrast, some microorganisms will also turn the Bile Esculin Agar plate dark red, but because of a low pH associated with bacteria metabolism. For such microorganisms, the red coloration is limited to the zone where the microorganism is growing.

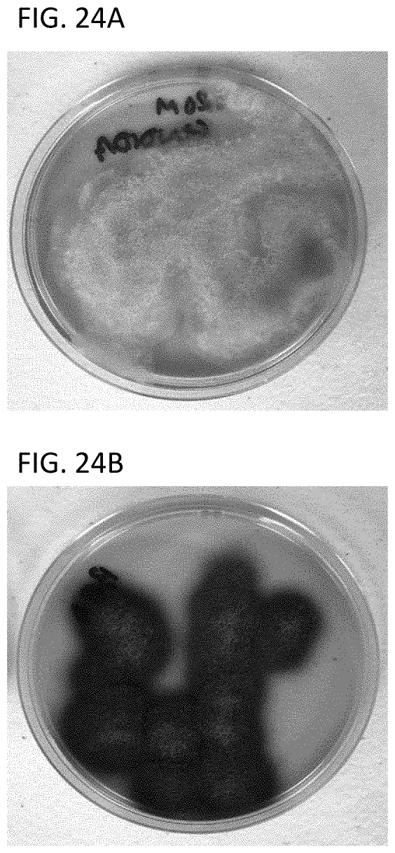

Examples of this include the Mucorales fungal organisms, with one example depicted in FIG. 24. Mucorales growth can be recognized by the red coloration on the Bile Esculin plate, and distinct from the red coloration from Enterococcus organisms (because the red coloration is limited to the zone of growth of the fungal organism), the fibrous appearance on the plate and from the absence of any coloration on the CromCandida plate (Candida organisms result in some coloration on the CromCandida plate).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1-2. (canceled)

3. A method of assessing whether or not a specific microorganism is present in a biological fluid, comprising:
(a) mixing the biological fluid with a reagent that is preselected to produce a specific response when the test microorganism is present:
(b) contact the biological fluid and reagent with a detection system that measures its optical absorbance at multiple wavelengths;
(c) measure a reference positive control optical absorption spectrum at an initial time using a known sample that contains the microorganism mixed with the reagent and a negative control optical absorption spectrum using a known sample that does not contain the microorganism mixed with the reagent;
(d) measure a “test” optical absorption spectrum from the biological fluid and reagent;
(d) generating a plurality of Rayleigh-corrected spectra by correcting one or more of the optical absorption spectra from the positive control, the negative control and the test sample;
(e) determining whether or not the bacteria in the positive control is present in the biological fluid by comparing the spectra from the test sample with the spectra of the positive and negative controls.

4. The method according to claim 3, wherein the method comprises one or more of:
determining if certain absorption peaks are present in the test sample by estimating if the absorption at the associated wavelength rises above a threshold;
determining if the maximum in the absorbance vs wavelength profile for the test sample is within a preset range associated with the positive control.
determining if the absorbance ratios at two wavelengths are within a range associated with the positive control;
determining if the absorbance at predetermined wavelengths exceeds a present threshold.
determining if the time dependent absorbance at particular wavelengths exceeds a preset threshold for a period of time;
determining that the dependent absorbance at particular wavelengths exceeds a preset threshold for a period of time and reverts to values below the preset threshold; and
reporting whether or not bacteria was determined to be present in the biological fluid.

5-11. (canceled)

12. The method of claim 3, wherein correcting the subsequent optical absorption spectrums for contributions from Rayleigh scattering comprises for each of the plurality of subsequent optical absorption spectrums:
(a) generating a change spectrum by subtracting the reference optical absorption spectrum from the subsequent optical absorption spectrum;
(b) generating a fit spectrum by fitting the change spectrum to a power function;
(c) generating a difference spectrum by subtracting the fit spectrum from the change spectrum;
(d) generating an adjusted spectrum by selecting:
points from the change spectrum for wavelengths wherein the difference spectrum is less than or equal to zero, and
points from the fit spectrum for wavelengths wherein the difference spectrum is greater than zero;
(e) repeating steps (a)-(c) zero or more times, wherein the most recent adjusted spectrum is used in place of the change spectrum if the steps are repeated, wherein the final adjusted spectrum is a Rayleigh profile; and
(f) generating a Rayleigh-corrected spectrum by subtracting the Rayleigh profile from the subsequent optical absorption spectrum.

13. The method of claim 12, wherein the microorganisms are bacteria.

14. The method of claim 13, wherein the power function has an order of −2, −3, or −4, wherein the order can be same or different between each of the optional repetitions.

15. The method of claim 12, wherein steps (a)-(c) are repeated 1 time, 2 times, or 3 times.

16. The method of claim 15, wherein the determining comprises comparing the rate of change in the two-dimensional plot of the biological fluid to a preset threshold.

17. The method of claim 16, wherein the detection component changes its optical absorbance in response a change in pH.

18. The method of claim 17, wherein the detection component changes its optical absorbance in response to an enzyme produced by a bacteria.

19. The method of claim 18, wherein the enzyme is a urease enzyme, wherein the contacting step further comprises contacting the fluid with urea.

20-21. (canceled)

22. A method of assessing the effect of a pharmaceutical drug on a microorganism, comprising
(i) for both a first fluid comprising the microorganism and the pharmaceutical drug and for a second fluid comprising the microorganism and lacking the pharmaceutical drug:
(a) contact the fluid with a detection component that changes its optical absorbance in response to a metabolic product of the microorganisms;
(b) measure a reference optical absorption spectrum at an initial time;
(c) measure a plurality of subsequent optical absorption spectrums at subsequent times;
(d) generating a plurality of Rayleigh-corrected spectrums by correcting the subsequent optical absorption spectrums for contributions from Rayleigh scattering;
(e) creating a two-dimensional plot using the Rayleigh-corrected spectrums, wherein one axis of the plot is time since the reference spectrum, wherein one axis of the plot is the change in absorbance at a particular wavelength or in a particular wavelength range since the reference spectrum; and
(ii) determining the effect of the pharmaceutical drug on the microorganism by comparing the two-dimensional for the first fluid to the two-dimensional plot for the second fluid.

23. (canceled)

24. A method of assessing the presence of a microorganism, the method comprising:
for a series of test samples that comprise all the test biological fluid with the unknown microorganism at the unknown concentration, a reagent media that supports microorganism growth and generates optical absorption, and a candidate antibiotic or pharmaceutical drug present at a series of concentrations that start at a high concentration above the resistant breakpoint and decreasing in factors of 2 such that the lowest concentration is below the susceptible breakpoint;
contacting the test samples with a detection component that changes its optical absorbance in response to a metabolic product of the microorganisms;
measuring a reference optical absorption spectrum at an initial time associated with a positive and a negative control, wherein the positive control include test samples comprises the microorganism present at a plurality of predetermined concentrations;
determining the time required to detect microorganism presence, and creating a master curve of time versus concentration of microorganism in the positive control;
measure a plurality of optical absorption spectrums at subsequent times from all the test samples;
generating a plurality of Rayleigh-corrected spectrums by correcting the subsequent optical absorption spectrums for contributions from Rayleigh scattering; and
determining the presence of the microorganism by comparing the test samples with positive and negative controls.

25. The method according to claim 24, wherein the method further comprises determining the concentration of the microorganism in the test sample by comparing the time required to determine microorganism presence with a master curve.

26. The method of assessing an effect of a pharmaceutical drug on an unknown microorganism present in a biological fluid, according to claim 24, wherein the method further comprises:
creating a set of samples wherein the concentration of the candidate pharmaceutical drug varies from a high concentration above the resistant breakpoint to a low concentration below the susceptible breakpoint;
plotting the time required to determine microorganism presence versus the concentration of the pharmaceutical drug from all the known samples that differ only in the concentration of the pharmaceutical drug; and
thresholding the concentration at which microorganism concentration does not change significantly from starting values.

27. The method according to claim 24, wherein the method further comprises determining a minimum inhibitory concentration (MIC) by determining the threshold concentration at which the time required for determining microorganism presence increases by a preset factor above the baseline value.

28. The method according to claim 27, wherein the method further comprises correcting the MIC for a standard pathogen concentration by using the concentration of the microorganism in the test sample determined by comparing the time required to determine microorganism presence with the master curve.

29. The method according to claim 27, wherein the method further comprises correcting the MIC for a standard pathogen concentration by using the concentration of the microorganism in the test sample determined by the threshold concentration at which the time required for determining microorganism presence increases by a preset factor above the baseline value.

30. The method according to claim 27, wherein the method further comprises correcting the MIC for a standard pathogen concentration by a predetermined master curve of the variation of MIC with pathogen concentration vs the absolute value of the estimated MIC.

31. The method according to claim 24, wherein the method further comprises characterizing a resistant, susceptible or intermediate status of the microorganism by comparing it against predetermined breakpoints.

32-33. (canceled)

34. The method according to claim 24, wherein the method further comprises performing steps (i)-(iii) for a third fluid comprising the microorganism and the pharmaceutical drug at a concentration different than the pharmaceutical drug concentration in the first fluid.

35-52. (canceled)
